(19) United States
(12) Patent Application Publication
(10) Pub. No.: US 2008/0253993 A1
(43) Pub. Date: Oct. 16, 2008

(54) Immunogenic Egfr Peptides Comprising Foreign T Cell Stimulating Epitope

(75) Inventors: Lucilla Steinaa, Horsholm (DK); Valery Renard, Horsholm (DK)

Correspondence Address: BIRCH STEWART KOLASCH & BIRCH, PO BOX 747, FALLS CHURCH, VA, 22040-0747, US

(73) Assignee: Pharmexa A/S, Horsholm (DK)

(21) Appl. No.: 11/909,983

(22) PCT Filed: Mar. 30, 2006
(86) PCT No.: PCT/DK2006/000184

§ 371 Date: Dec. 6, 2007

Related U.S. Application Data
(60) Provisional application No. 60/667,441, filed on Mar. 31, 2005.

(30) Foreign Application Priority Data
Mar. 31, 2005 (DK) PA200500448

(51) Int. Cl. A61K 45/00; A61K 39/00; A61K 38/21; A61K 9/127; C07K 14/00; C12N 15/11; C12N 15/00; C12N 1/20; C12N 15/74; A61P 37/00

(52) U.S. Cl. 424/85.2; 424/184.1; 424/85.5; 424/199.1; 424/200.1; 424/450; 530/350; 536/23.5; 435/320.1; 435/252.3; 435/471

(57) ABSTRACT

Disclosed is a method for inducing an immune response against autologous Epidermal Growth Factor Receptor (EGFR) in humans. The method comprises effecting uptake by antigen presenting cells of epitopes (preferably all) from the extracellular portion of human EGFR and of at least one non-human T helper epitope (T_{H} epitope) so as to induce antibodies against EGFR. Immunization may be accomplished by protein vaccination, nucleic acid vaccination and live or viral vaccination. The immune response is useful in treatment of malignancies. Also disclosed are modified EGFR proteins and expression plasmids useful for the immunization method as well as recombinant gene technology tools such as nucleic acids, vectors and host cells.

### FIELD OF THE INVENTION

The present invention relates to the field of active specific immunotherapy. More specifically, the present invention relates to novel immunogenic variants of epithelial growth factor receptor (EGFR) and to methods for immunological targeting of cells expressing DNA which encodes EGFR.

### BACKGROUND OF THE INVENTION

EGFR is a 170 kDa transmembrane protein. The extracellular part is 110 kDa including carbohydrates and 70 kDa when it is deglycosylated. The extracellular part consists of four domains: L1 (domain I), CR1 (domain II), L2 (domain III), CR2 (domain IV). CR1 and CR2 are cysteine-rich domains that have a strong structural function. L1 and L2 are right handed P-helices, which is a helical structure formed by β-strands. The major ligands EGF and TGF-alpha bind to L2. This is also the domain to which several inhibiting mAb's bind.

EGFR induces cell differentiation and proliferation upon binding of one of its ligands. Ligand binding induces a conformational change in the EGFR monomer, which leads to dimerization with another EGFR or with another molecule belonging to the EGFR-family (HER-2, HER-3 or HER-4). This leads to autophosphorylation and increased activity of the tyrosine kinase. Five different ligands are known: epidermal growth factor (EGF), transforming growth factor-α (TGF-α), amphiregulin (AR), Heparin-binding EGF-like growth factor (HB-EGF) and betacellulin (BTC). Following ligand binding, the receptor is internalized and in case EGF binds, the receptor is degraded. In case TGF-α binds, it is recycled.

Overexpression of EGFR has been observed in many types of human cancers such as breast, head&neck squamous carcinomas, colorectal cancers, ovarian and non-small-cell lung cancers. Overexpression has been linked with poor prognosis in a number of cancers, including breast cancer, gliomas, squamous carcinomas and laryngeal cancers. In other cancers, there is controversy to which extent EGFR can be used as a prognostic marker, e.g. non-small-cell lung cancer. Besides overexpression of the normal EGFR, a variant that lacks amino acids 6-273 is expressed in a high proportion of breast and ovarian cancers and to a lesser extent in lung cancers. This variant has no ligand binding activity but is constitutively active. It is not expressed in normal tissues.

The potential of EGFR as a target for cancer immunotherapy has been verified both by numerous animal studies and also by clinical studies. Several monoclonal antibodies, tyrosine kinase inhibitors and antisense oligonucleotides inhibit growth of human cancer cell lines in nude mice or SCID mice. In clinical studies, a monoclonal antibody (C225) developed by Imclone has proven to be effective in patients with head&neck squamous cancer and is currently in phase III. A small molecule tyrosine kinase inhibitor “Iressa” has also shown to be effective in clinical trials.

### Tyrosine Kinase Inhibitors

Iressa® is a small molecule, a reversible inhibitor of the EGFR tyrosine kinase, the efficacy of which has been evaluated in several clinical trials, with the best results in a trial with 99 non-small-cell lung carcinoma patients. 8 patients had partial responses lasting 1-16 months. 2 patients had regression of nonmesurable evaluable disease and one third of patients had long-lasting stable disease.

Tarceva® is a quinazoline derivative, an orally active EGFR tyrosine kinase inhibitor. It is currently in phase I, II and III trials, heading for both frontline and refractory therapy for various cancer indications. Tarceva® is a structural homologue of Iressa®.

These tyrosine kinase inhibitors have shown good efficacy in the clinical trial. They inhibit EGFR by competing with ATP binding in the ATP binding pocket. It is possible to make specific inhibitors in this category, but the specificity is a relative phenomena. For instance, Iressa® is also known to inhibit HER-2 but to a lower extent.

There appear to be some acne-like dermal effects, which are also observed with monoclonal antibodies, cf. below. This side effect is directly attributable to the targeting of EGFR. The grade 3-4 diarrhea that has been observed as an adverse effect using the tyrosine kinase inhibitors, has not been observed with the monoclonal antibodies. This side effect may therefore not be related to the targeting of EGFR but is probably related to the more unspecific inhibition of different tyrosine kinases by the small inhibitors.

### Monoclonal Antibodies

ImClone Inc. has developed a mouse-human chimeric monoclonal antibody, Cetuximab®, which has completed a phase III clinical trial. This monoclonal antibody is a chimeric antibody and therefore not completely human. This leads to human anti-chimeric responses in about 3% of the patients.

Cetuximab® seems to be especially efficient in combination with radiation and chemotherapy. A phase I study in head&neck patients receiving Cetuximab in combination with cisplatin showed major responses in 6 out of 9 patients (67%) including 2 complete remissions. Likewise, in another phase I study involving advanced head&neck cancer patients with Cetuximab® in combination with radiation, the response rate was 100% and 13 of 15 patients achieved complete remissions. The expected complete plus partial remission with radiation alone is 50-60%.

### SUMMARY OF THE INVENTION

The present invention contemplates the preparation of an EGFR vaccine molecule that elicits autoreactive antibodies, which in turn inhibit growth of EGFR-overexpressing cancers. These antibodies are contemplated to affect tumor growth by inhibiting ligand binding, dimerization, opsonization and induction of internalization or apoptosis. Apart from the normal overexpressed EGFR, a deletional mutant of EGFR (EGFRvIII) is expressed in many of these cancers, and also this particular mutant is targeted.

Vaccine molecules are primarily based on the extracellular domain of the transmembrane receptor molecule, since antibodies are believed to be the important biological effector molecules.

One family of immunonic molecules are based on the full-length extracellular domain, which is a natural variant of EGFR. This molecule has previously been expressed in insect cells. Another family will be based on the first 75% N-terminal amino acids of the extracellular domain, a molecule that has previously been expressed well.

Variants are tested immunologically by transfer experiments of antibodies/cells to nude mice xenografted with a human tumor cell line. Variants will be tested in an in vitro inhibitory binding assays, a tumor inhibitory assays and maybe also in assays for tyrosine kinase activity and internalization.

It is believed that the present approach is advantageous over tyrosine kinase inhibitors and monoclonal antibodies regarding efficacy, because of the large panel of different biological effects they potentially produce. Furthermore, the polyclonal antibodies that are induced will react with the deletional mutant EGFRvIII. This is not the case for several of the monoclonal antibodies that have been developed to target normal EGFR.

Further, the present approach is believed to present fewer adverse effects, since it does not suffer from the infusion related side effects known with mAbs, and since it is expected not to cause diarrhea in a fraction of patients as does the tyrosine kinase inhibitors.

An anti-EGFR vaccine is therefore an advantage compared to small inhibitors and monoclonal antibodies because of the abilities to attack the cancer cell and EGFR at many different points. A polyclonal antibody response has also the capability of reacting with the truncated variant found in many cancers. Further, because of the high specificity of antibodies, this may create fewer side effects compared to tyrosine kinase inhibitors, which are somewhat more promiscuous. Compared to monoclonal antibodies, a vaccine is very easy to administrate. Furthermore, no immune response will develop against the therapeutic antibodies generated by a vaccine.

### LEGENDS TO THE FIGURE

FIG. 1: EGFR wt-templates.

The full-length extracellular domain (upper) is the basis for one family of constructs. The truncated extracellular domain (1-501) is the basis for a smaller family of constructs. Ligand-binding domains (L) and cysteine rich (CR) domains are indicated.

FIG. 2: Anti-tumor effect of anti-EGFR-5D antiserum in a Xenograft model.

One day prior to tumor challenge with 1.106 A431 tumor cells, Nude mice were injected intraperitoneally with 400 μl of antiserum. The number of mice developing tumors is indicated in parenthesis for each treatment group. Black dots indicate number of mice developing tumors after having received hyperimmune serum from mice immunized with the EGFR-5D variant, diamonds indicate number of mice developing tumours after having received a control immune serum, and triangles indicate number of untreated mice developing tumours.

### DETAILED DISCLOSURE OF THE INVENTION

### Definitions

In the following, a number of terms used in the present specification and claims will be defined and explained in detail in order to clarify the metes and bounds of the invention.

The term “immunogen” in the present context refers to an agent (a substance or a composition of matter) that induces a specific immune response against the immunogen in a host which has been immunized therewith. It will be understood that certain molecules (e.g. traditional small haptens or self-proteins that are tolerated in the autologous host) are incapable of inducing a specific immune response. However, some self-proteins are, when formulated in very strong immunologic adjuvants, capable of inducing a specific immune response in spite of the normally tolerant state of the immunized animal. In such a context, the “immunogen” is therefore the composition of matter (self-protein with adjuvant) and not just a single molecule.

The terms “T-lymphocyte” and “T-cell” will be used interchangeably for lymphocytes of thymic origin which are responsible for various cell mediated immune responses as well as for helper activity in the humeral immune response. Likewise, the terms “B-lymphocyte” and “B-cell” will be used interchangeably for antibody-producing lymphocytes.

An “EGFR polypeptide” is herein intended to denote polypeptides having the amino acid sequence of the above-discussed EGFR proteins derived from humans and other mammals (or truncates thereof sharing a substantial amount of B-cell epitopes with intact EGFR), but also polypeptides having the amino acid sequence identical to xeno-analogues of these proteins isolated from other species are embraced by the term; included in the term is both the membrane bound EGFR polypeptide as well as soluble fragments of the EGFR and the extracellular domain of EGFR. Also un-glycosylated forms of EGFR which are prepared in prokaryotic system are included within the boundaries of the term as are forms having varying glycosylation patterns due to the use of e.g. yeasts or other non-mammalian eukaryotic expression systems.

It should, however, be noted that when using the term “an EGFR polypeptide” it is intended that the polypeptide in question is normally non-immunogenic when presented to the animal to be treated. In other words, the EGFR polypeptide is a self-protein or is a xeno-analogue of such a self-protein which will not normally give rise to an immune response against EGFR of the animal in question.

An “EGFR analogue” is an EGFR polypeptide which has been subjected to changes in its primary structure. Such a change can e.g. be in the form of fusion of an EGFR polypeptide to a suitable fusion partner (i.e. a change in primary structure exclusively involving C- and/or N-terminal additions of amino acid residues) and/or it can be in the form of insertions and/or deletions and/or substitutions in the EGFR polypeptide's amino acid sequence. Also encompassed by the term are derivatized EGFR molecules, cf. the discussion below of modifications of EGFR.

It should be noted that the use as a vaccine in a human of e.g. a canine analogue of human EGFR could be imagined to produce the desired immunity against EGFR. Such use of a xeno-analogue for immunization is also considered to be a “EGFR analogue” as defined above.

When using the abbreviation “EGFR” herein, this is intended as a reference to the amino acid sequence of wildtype EGFR (also denoted “EGFR” and “EGFR-wt” herein). In cases where a DNA construct includes information encoding a leader sequence or other material, this will normally be clear from the context.

The term “polypeptide” is in the present context intended to mean both short peptides of from 2 to 10 amino acid residues, oligopeptides of from 11 to 100 amino acid residues, and polypeptides of more than 100 amino acid residues. Furthermore, the term is also intended to include proteins, i.e. functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups. Also, the term “polyamino acid” is an equivalent to the term “polypeptide”.

The term “subsequence” means any consecutive stretch of at least 3 amino acids or, when relevant, of at least 3 nucleotides, derived directly from a naturally occurring EGFR amino acid sequence or nucleic acid sequence, respectively.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method of the invention all harbour substantially the same EGFR allowing for immunization of the animals with the same immunogen(s). If, for instance, genetic variants of EGFR exist in different human population it may be necessary to use different immunogens in these different populations in order to be able to break the autotolerance towards EGFR in each population. It will be clear to the skilled person that an animal in the present context is a living being which has an immune system. It is preferred that the animal is a vertebrate, such as a mammal.

By the term “in vivo down-regulation of EGFR activity” is herein meant reduction in the living organism of the number of interactions between EGFR and its natural binding partners and ligands (or between EGFR and other possible biologically important binding partners for this molecule). The down-regulation can be obtained by means of several mechanisms: Of these, simple interference with the active site in EGFR by antibody binding is the most simple. However, it is also within the scope of the present invention that the antibody binding results in removal of EGFR-carrying cells by secondary immunological mechanisms (such as complement activation and killing of cells by NK cells. Also within the scope of the present invention is the killing of EGFR expressing cells by cytotoxic T lymphocytes.

The expression “effecting presentation . . . to the immune system” is intended to denote that the animal's immune system is subjected to an immunogenic challenge in a controlled manner. As will appear from the disclosure below, such challenge of the immune system can be effected in a number of ways of which the most important are vaccination with polypeptide containing “pharmaccines” (i.e. a vaccine which is administered to treat or ameliorate ongoing disease) or nucleic acid “pharmaccine” vaccination. The important result to achieve is that immune competent cells in the animal are confronted with the antigen in an immunologically effective manner, whereas the precise mode of achieving this result is of less importance to the inventive idea underlying the present invention.

The term “immunogenically effective amount” has its usual meaning in the art of immunology, i.e. an amount of an immunogen which is capable of inducing an immune response which significantly engages molecules which share immunological features with the immunogen.

When using the expression that the EGFR has been “modified” is herein meant a chemical modification of the polypeptide which constitutes the backbone of EGFR. Such a modification can e.g. be derivatization (e.g. alkylation, acylation, esterification etc.) of certain amino acid residues in the EGFR sequence, but as will be appreciated from the disclosure below, the preferred modifications comprise changes of (or additions to) the primary structure of the EGFR amino acid sequence.

When discussing “autotolerance towards EGFR” it is understood that since EGFR is a self-protein in the population to be vaccinated, normal individuals in the population do not mount an immune response against EGFR; it cannot be excluded, though, that occasional individuals in an animal population might be able to produce antibodies against native EGFR, e.g. as part of an autoimmune disorder. At any rate, an animal will normally only be autotolerant towards its own EGFR, but it cannot be excluded that EGFR analogues derived from other animal species or from a population having a different EGFR phenotype would also be tolerated by said animal.

A “foreign T-cell epitope” (or: “foreign T-lymphocyte epitope”) is a peptide which is able to bind to an MHC molecule and which stimulates T-cells in an animal species. Preferred foreign T-cell epitopes in the invention are “promiscuous” (also known as “universal”) epitopes, i.e. epitopes which bind to a substantial fraction of a particular class of MHC molecules in an animal species or population. Only a very limited number of such promiscuous T-cell epitopes are known, and they will be discussed in detail below. It should be noted that in order for the immunogens which are used according to the present invention to be effective in as large a fraction of an animal population as possible, it may be necessary to 1) insert several foreign T-cell epitopes in the same EGFR analogue or 2) prepare several EGFR analogues wherein each analogue has a different promiscuous epitope inserted. It should be noted also that the concept of foreign T-cell epitopes also encompasses use of cryptic T-cell epitopes, i.e. epitopes which are derived from a self-protein and which only exerts immunogenic behaviour when existing in isolated form without being part of the self-protein in question.

A “foreign T helper lymphocyte epitope” (a foreign T_{H} epitope) is a foreign T cell epitope which binds an MHC Class II molecule and can be presented on the surface of an antigen presenting cell (APC) bound to the MHC Class II molecule.

A “functional part” of a (bio)molecule is in the present context intended to mean the part of the molecule which is responsible for at least one of the biochemical or physiological effects exerted by the molecule. It is well-known in the art that many enzymes and other effector molecules have an active site which is responsible for the effects exerted by the molecule in question. Other parts of the molecule may serve a stabilizing or solubility enhancing purpose and can therefore be left out if these purposes are not of relevance in the context of a certain embodiment of the present invention. For instance it is possible to use certain cytokines as a modifying moiety in EGFR (cf. the detailed discussion below), and in such a case, the issue of stability may be irrelevant since the coupling to EGFR provides the stability necessary.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide a specific immune response against the immunogen, vaccination with the immunogen may or may not give rise to a specific immune response against the immunogen, but the combination of vaccination with immunogen and adjuvant induces a specific immune response against the immunogen which is stronger than that induced by the immunogen alone.

“Targeting” of a molecule is in the present context intended to denote the situation where a molecule upon introduction in the animal will appear preferentially in certain tissue(s) or will be preferentially associated with certain cells or cell types. The effect can be accomplished in a number of ways including formulation of the molecule in composition facilitating targeting or by introduction in the molecule of groups which facilitates targeting. These issues will be discussed in detail below.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

“Productive binding” means binding of a peptide to the MHC molecule (Class I or II) so as to be able to stimulate T-cells that engage a cell that present a peptide bound to the MHC molecule. For instance, a peptide bound to an MHC Class II molecule on the surface of an APC is said to be productively bound if this APC will stimulate a T_{H} cell that binds to the presented peptide-MHC Class II complex and, accordingly, a peptide bound to an MHC Class I molecule on the surface of a cell is said to be productively bound if activated CTLs that recognize the peptides are capable of exerting a cytotoxic effect on the cell.

### PREFERRED EMBODIMENTS OF ANTI-EGFR IMMUNIZATION

The present invention entails active immunization against EGFR. More precisely, the invention pertains to a method for inducing an immune response against autologous Epidermal Growth Factor Receptor (EGFR) in a human subject, the method comprising effecting uptake and processing by antigen presenting cells (APCs) in the subject of at least one modified EGFR polypeptide, said at least one modified EGFR polypeptide comprising
a substantial fraction of the B-cell epitopes from the extracellular portion of human EGFR, and
at least one non-human T helper epitope (T_{H} epitope), thereby inducing an antibody response that targets the autologous EGFR.

In one aspect, the active immunization is used to treat, ameliorate or reduce the risk of attracting neoplastic diseases such as cancer.

The EGFR polypeptide used as an immunogen in the method of the invention is thus a modified molecule wherein at least one change is present in the EGFR polypeptide amino acid sequence, since the chances of obtaining the all-important breaking of autotolerance towards EGFR is greatly facilitated that way. It should be noted that this does not exclude the possibility of using such a modified EGFR in formulations which further facilitate the breaking of autotolerance against EGFR, e.g. formulations containing certain adjuvants discussed in detail below.

It has been shown (in Dalum I et al., 1996, J. Immunol. 157: 4796-4804) that potentially self-reactive B-lymphocytes recognizing self-proteins are physiologically present in normal individuals. However, in order for these B-lymphocytes to be induced to actually produce antibodies reactive with the relevant self-proteins, assistance is needed from cytokine producing T-helper lymphocytes (T_{H}-cells or T_{H}-lymphocytes). Normally this help is not provided because T-lymphocytes in general do not recognize T-cell epitopes derived from self-proteins when presented by antigen presenting cells (APCs). However, by providing an element of “foreignness” in a self-protein (i.e. by introducing an immunologically significant modification), T-cells recognizing the foreign element are activated upon recognizing the foreign epitope on an APC (such as, initially, a mononuclear cell). Polyclonal B-lymphocytes (which are also specialised APCs) capable of recognising self-epitopes on the modified self-protein also internalise the antigen and subsequently presents the foreign T-cell epitope(s) thereof, and the activated T-lymphocytes subsequently provide cytokine help to these self-reactive polyclonal B-lymphocytes. Since the antibodies produced by these polyclonal B-lymphocytes are reactive with different epitopes on the modified polypeptide, including those which are also present in the native polypeptide, an antibody cross-reactive with the non-modified self-protein is induced. In conclusion, the T-lymphocytes can be led to act as if the population of polyclonal B-lymphocytes have recognised an entirely foreign antigen, whereas in fact only the inserted epitope(s) is/are foreign to the host. In this way, antibodies capable of cross-reacting with non-modified self-antigens are induced.

Several ways of modifying a peptide self-antigen in order to obtain improved breaking of autotolerance (in addition to the inclusion of foreign T helper epitopes) are known in the art. Hence, according to the invention, the modification can include that
at least one first moiety is introduced which effects targeting of the modified molecule to an antigen presenting cell (APC), and/or
at least one second moiety is introduced which stimulates the immune system, and/or
at least one third moiety is introduced which optimises presentation of the modified EGFR polypeptide to the immune system.

However, all these modifications should be carried out while maintaining a substantial fraction of the original B-lymphocyte epitopes in the extracellularly exposed parts of EGFR, since the B-lymphocyte recognition of the native molecule is thereby enhanced. Preferred variants therefore include deletions of all of or parts the intracellular parts of EGFR. The modified human EGFR polypeptide, on the other hand, typically comprises at least 60% of the 621 amino acids constituting the amino acid sequence of the extracellular domain of human EGFR, although higher percentages are preferred, such as at least 65, at least 75, at least 80, at least 85, at least 90, and at least 95% of the extracellular domain. In an embodiment, all known epitopes of the extracellular portion of autologous EGFR are present in the first analogue and in another embodiment, substantially all predicted epitopes of the extracellular portion of autologous EGFR are present in the at least first analogue. These two embodiments can be combined.

In one embodiment, side groups (in the form of foreign T-cell epitopes or the above-mentioned first, second and third moieties) are covalently or non-covalently introduced. This is intended to mean that stretches of amino acid residues derived from EGFR are derivatized without altering the primary amino acid sequence, or at least without introducing changes in the peptide bonds between the individual amino acids in the chain.

An alternative embodiment utilises amino acid substitution and/or deletion and/or insertion and/or addition (which may be effected by recombinant means or by means of peptide synthesis; modifications which involves longer stretches of amino acids can give rise to fusion polypeptides). One version of this embodiment is the technique described in WO 95/05849, which discloses a method for immunizing against self-proteins by immunising with analogues of the self-proteins wherein a number of amino acid sequence(s) has been substituted with a corresponding number of amino acid sequence(s), which each comprise a foreign immunodominant T-cell epitope, while at the same time maintaining the overall 3 dimensional structure of the self-protein in the analogue. For the purposes of the present invention, it is however sufficient if the modification (be it an amino acid insertion, addition, deletion or substitution) gives rise to a foreign T-cell epitope and at the same time preserves a substantial number of the B-cell epitopes in EGFR. However, in order to obtain maximum efficacy of the immune response induced, it is advantageous that the 3-dimenstional structure of at least the extracellularly exposed parts of EGFR is maintained in the modified molecule. This means that it is advantageous if modifications in the EGFR structure are made in a non-destructive way, e.g. in flexible loops or termini.

The following formula describes the EGFR constructs generally covered by the invention:

(MOD₁)ₛ₁(Eₑ₁)ₙ₁(MOD₂)ₛ₂(Eₑ₂)ₙ₂ . . . (MODₓ)ₛₓ(Eₑₓ)ₙₓ (I)
where Eₑ₁-Eₑₓ are x B-cell epitope containing subsequences of an EGFR polypeptide, which independently are identical or non-identical and which may contain or not contain foreign side groups, x is an integer≧3, n1-nx are x integers≧0 (at least one is ≧1), MOD₁-MODₓ are x modifications introduced between the preserved B-cell epitopes, and s₁-sₓ are x integers≧0 (at least one is ≧1 if no side groups are introduced in the Eₑₓ sequences). Thus, given the general functional restraints on the immunogenicity of the constructs, the invention allows for all kinds of permutations of the original sequence of the EGFR polypeptide, and all kinds of modifications therein. Thus, included in the invention are modified EGFR polypeptides obtained by omission of parts of the sequence of the EGFR polypeptide, which e.g. exhibit adverse effects in vivo and thus could give rise to undesired immunological reactions.

One embodiment of the invention utilises multiple presentations of B-lymphocyte epitopes of the EGFR polypeptide (i.e. formula I wherein at least one B-cell epitope is present in two positions). This effect can be achieved in various ways, e.g. by simply preparing fusion polypeptides comprising the structure (EGFR polypeptide)ₘ, where m is an integer≧2 and then introduce the modifications discussed herein in at least one of the EGFR sequences. Or, It is possible to couple multiple EGFR peptides or polypeptides to a carrier backbone, whereby multiple presentations of identical EGFR B-cell epitopes are achieved. It is hence preferred that the modifications introduced includes at least one duplication of a B-lymphocyte epitope and/or the introduction of a hapten. These embodiments including multiple presentations of selected epitopes are especially preferred in situations where merely minor parts of the EGFR polypeptide are useful as constituents in a vaccine agent.

As mentioned above, the introduction of a foreign T-cell epitope can be accomplished by introduction of at least one amino acid insertion, addition, deletion, or substitution. Of course, the normal situation will be the introduction of more than one change in the amino acid sequence (e.g. insertion of or substitution by a complete T-cell epitope) but the important goal to reach is that the analogue, when processed by an antigen presenting cell (APC) such as a macrophage or a dendritic cell, will give rise to such a foreign immunodominant T-cell epitope being presented in context of an MCH Class II molecule on the surface of the APC. Thus, if the amino acid sequence of the EGFR polypeptide in appropriate positions comprises a number of amino acid residues which can also be found in a foreign T_{H} epitope then the introduction of a foreign T_{H} epitope can be accomplished by providing the remaining amino acids of the foreign epitope by means of amino acid insertion, addition, deletion and substitution. In other words, it is not necessary to introduce a complete T_{H} epitope by insertion or substitution in order to fulfil the purpose of the present invention.

The number of amino acid insertions, deletions, substitutions or additions is typically at least 2, such as 3, 4, 5, 6, 7, 8, 9, 10, 11, 12, 13, 14, 15, 16, 17, 18, 19, 20, 21 and 25 insertions, substitutions, additions or deletions. The number of amino acid insertions, substitutions, additions or deletions does normally not exceed 150, so that at most 100, at most 90, at most 80, and at most 70 changes are introduced. The number of substitutions, insertions, deletions, or additions does in some embodiments not exceed 60, and in particular the number does not exceed 50 or even 40 in these embodiment. In certain embodiments, the number is not more than 30. With respect to amino acid additions, it should be noted that these, when the resulting construct is in the form of a fusion polypeptide, is often considerably higher than 150.

Some embodiments of the invention include modification by introducing at least one foreign immunodominant T-cell epitope. It will be understood that the question of immune dominance of a T-cell epitope depends on the animal species in question. As used herein, the term “immunodominance” simply refers to epitopes which in the vaccinated individual/population gives rise to a significant immune response, but it is a well-known fact that a T-cell epitope which is immunodominant in one individual/population is not necessarily immunodominant in another individual of the same species, even though it may be capable of binding MHC-II molecules in the latter individual. Hence, for the purposes of the present invention, an immune dominant T-cell epitope is a T-cell epitope which will be effective in providing T-cell help when present in an antigen. Typically, immune dominant T-cell epitopes has as an inherent feature that they will substantially always be presented bound to an MHC Class II molecule, irrespective of the polypeptide wherein they appear.

Another important point is the issue of MHC restriction of T-cell epitopes. In general, naturally occurring T-cell epitopes are MHC restricted, i.e. a certain peptides constituting a T-cell epitope will only bind effectively to a subset of MHC Class II molecules. This in turn has the effect that in most cases the use of one specific T-cell epitope will result in a vaccine component which is only effective in a fraction of the population, and depending on the size of that fraction, it can be necessary to include more T-cell epitopes in the same molecule, or alternatively prepare a multi-component vaccine wherein the components are variants of the EGFR polypeptide which are distinguished from each other by the nature of the T-cell epitope introduced.

If the MHC restriction of the T-cells used is completely unknown (for instance in a situation where the vaccinated animal has a poorly defined MHC composition), the fraction of the population covered by a specific vaccine composition can be determined by means of the following formula

$${f}_{population} = 1 - {\prod }_{i = 1}^{n} ( 1 - {p}_{i} ) \qquad ( II )$$

where pᵢ is the frequency in the population of responders to the i^{th} foreign T-cell epitope pre-sent in the vaccine composition, and n is the total number of foreign T-cell epitopes in the vaccine composition. Thus, a vaccine composition containing 3 foreign T-cell epitopes having response frequencies in the population of 0.8, 0.7, and 0.6, respectively, would give

1−0.2×0.3×0.4=0.976
i.e. 97.6 percent of the population will statistically mount an MHC-II mediated response to the vaccine.

The above formula does not apply in situations where a more or less precise MHC restriction pattern of the peptides used is known. If, for instance a certain peptide only binds the human MHC-II molecules encoded by HLA-DR alleles DR1, DR3, DR5, and DR7, then the use of this peptide together with another peptide which binds the remaining MHC-II molecules encoded by HLA-DR alleles will accomplish 100% coverage in the population in question. Likewise, if the second peptide only binds DR3 and DR5, the addition of this peptide will not increase the coverage at all. If one bases the calculation of population response purely on MHC restriction of T-cell epitopes in the vaccine, the fraction of the population covered by a specific vaccine composition can be determined by means of the following formula:

$${f}_{population} = 1 - {\prod }_{j = 1}^{3} {( 1 - {ϕ}_{j} )}^{2} \qquad ( III )$$

wherein φⱼ is the sum of frequencies in the population of allelic haplotypes encoding MHC molecules which bind any one of the T-cell epitopes in the vaccine and which belong to the j^{th} of the 3 known HLA loci (DP, DR and DQ); in practice, it is first determined which MHC molecules will recognize each T-cell epitope in the vaccine and thereafter these are listed by type (DP, DR and DQ)—then, the individual frequencies of the different listed allelic haplotypes are summed for each type, thereby yielding φ₁, φ₂, and φ₃.

It may occur that the value pᵢ in formula II exceeds the corresponding theoretical value nᵢ:

$${π}_{i} = 1 - {\prod }_{j = 1}^{3} {( 1 - {v}_{j} )}^{2} \qquad ( IV )$$

wherein uⱼ is the sum of frequencies in the population of allelic haplotype encoding MHC molecules which bind the i^{th} T-cell epitope in the vaccine and which belong to the j^{th} of the 3 known HLA loci (DP, DR and DQ). This means that in 1−nᵢ of the population is a frequency of responders of f_{residual}_{_{—}}ᵢ=(pᵢ−nᵢ)/(1−nᵢ). Therefore, formula III can be adjusted so as to yield formula V:

$${f}_{population} = 1 - {\prod }_{j = 1}^{3} {( 1 - {ϕ}_{j} )}^{2} + ( 1 - {\prod }_{i = 1}^{n} ( 1 - {f}_{residual\_i} ) ) \qquad ( V )$$

where the term 1-f_{residual-i} is set to zero if negative. It should be noted that formula V requires that all epitopes have been haplotype mapped against identical sets of haplotypes.

Therefore, when selecting T-cell epitopes to be introduced in the EGFR analogue, it is important to include all knowledge of the epitopes which is available: 1) The frequency of responders in the population to each epitope, 2) MHC restriction data, and 3) frequency in the population of the relevant haplotypes.

There exists a number of naturally occurring “promiscuous” T-cell epitopes which are active in a large proportion of individuals of an animal species or an animal population and these are preferably introduced in the vaccine thereby reducing the need for a very large number of different analogues in the same vaccine.

The promiscuous epitope can according to the invention be a naturally occurring human T-cell epitope such as epitopes from tetanus toxoid (e.g. the P2 and P30 epitopes exemplified herein, cf. SEQ ID NOs: 2 and 3, respectively), diphtheria toxoid, Influenza virus hemagluttinin (HA), and P. falciparum CS antigen.

Over the years a number of other promiscuous T-cell epitopes have been identified. Especially peptides capable of binding a large proportion of HLA-DR molecules encoded by the different HLA-DR alleles have been identified and these are all possible T-cell epitopes to be introduced in the analogues used according to the present invention. Cf. also the epitopes discussed in the following references which are hereby all incorporated by reference herein: WO 98/23635 (Frazer I H et al., assigned to The University of Queensland); Southwood S et al., 1998, J. Immunol. 160: 3363-3373; Sinigaglia F et al., 1988, Nature 336: 778-780; Chicz R M et al., 1993, 3. Exp. Med. 178: 27-47; Hammer J et al., 1993, Cell 74: 197-203; and Falk K et al., 1994, Immunogenetics 39: 230-242. The latter reference also deals with HLA-DQ and -DP ligands. All epitopes listed in these 5 references are relevant as candidate natural epitopes to be used in the present invention, as are epitopes which share common motifs with these.

Alternatively, the epitope can be any artificial T-cell epitope which is capable of binding a large proportion of MHC Class II molecules. In this context the pan DR epitope peptides (“PADRE”) described in WO 95/07707 and in the corresponding paper Alexander J et al., 1994, Immunity 1: 751-761 (both disclosures are incorporated by reference herein) are interesting candidates for epitopes to be used according to the present invention. It should be noted that the most effective PADRE peptides disclosed in these papers carry D-amino acids in the C- and N-termini in order to improve stability when administered. However, the present invention primarily aims at incorporating the relevant epitopes as part of the modified EGFR polypeptide, which should then subsequently be broken down enzymatically inside the lysosomal compartment of APCs to allow subsequent presentation in the context of an MHC-II molecule and therefore it is not expedient to incorporate D-amino acids in the epitopes used in the present invention.

One especially preferred PADRE peptide is the one having the amino acid sequence SEQ ID NO: 4 (AKFVAAWTLKAAA) or an immunologically effective subsequence thereof. This and other epitopes having the same lack of MHC restriction are preferred T-cell epitopes which should be present in the analogues used in the inventive method. Such super-promiscuous epitopes will allow for the simplest embodiments of the invention wherein only one single modified EGFR polypeptide is presented to the vaccinated animal's immune system.

As mentioned above, the modification of the EGFR polypeptide can also include the introduction of a first moiety which targets the modified EGFR polypeptide to an APC or a B-lymphocyte. For instance, the first moiety can be a specific binding partner for a B-lymphocyte specific surface antigen or for an APC specific surface antigen. Many such specific surface antigens are known in the art. For instance, the moiety can be a carbohydrate for which there is a receptor on the B-lymphocyte or the APC (e.g. mannan or mannose). Alternatively, the second moiety can be a hapten. Also an antibody fragment which specifically recognizes a surface molecule on APCs or lymphocytes can be used as a first moiety (the surface molecule can e.g. be an FCγ receptor of macrophages and monocytes, such as FCγRI or, alternatively any other specific surface marker such as CD40 or CTLA-4). It should be noted that all these exemplary targeting molecules can be used as part of an adjuvant also, cf. below.

As an alternative or supplement to targeting the modified EGFR polypeptide to a certain cell type in order to achieve an enhanced immune response, it is possible to increase the level of responsiveness of the immune system by including the above-mentioned second moiety which stimulates the immune system. Typical examples of such second moieties are cytokines, and heat-shock proteins or molecular chaperones, as well as effective parts thereof.

Suitable cytokines to be used according to the invention are those which will normally also function as adjuvants in a vaccine composition, i.e. for instance interferon γ (IFN-γ), interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF); alternatively, the functional part of the cytokine molecule may suffice as the second moiety. With respect to the use of such cytokines as adjuvant substances, cf. the discussion below.

According to the invention, suitable heat-shock proteins or molecular chaperones used as the second moiety can be HSP70 (heat shock protein 70), HSP90 (heat shock protein 90), HSC70 (heat shock cognate protein 70), GRP94 (also known as gp96, cf. Wearsch P A et al. 1998, Biochemistry 37: 5709-19), and CRT (calreticulin).

Alternatively, the second moiety can be a toxin, such as listeriolycin (LLO), lipid A and heat-labile enterotoxin. Also, a number of mycobacterial derivatives such as MDP (muramyl dipeptide), CFA (complete Freund's adjuvant) and the trehalose diesters TDM and TDE are interesting possibilities.

Also the possibility of introducing a third moiety which enhances the presentation of the modified EGFR polypeptide to the immune system is an important embodiment of the invention. The art has shown several examples of this principle. For instance, it is known that the palmitoyl lipidation anchor in the Borrelia burgdorferi protein OspA can be utilised so as to provide self-adjuvating polypeptides (cf. e.g. WO 96/40718)—it seems that the lipidated proteins form up micelle-like structures with a core consisting of the lipidation anchor parts of the polypeptides and the remaining parts of the molecule protruding there from, resulting in multiple presentations of the antigenic determinants. Hence, the use of this and related approaches using different lipidation anchors (e.g. a myristyl group, a myristyl group, a farnesyl group, a geranyl-geranyl group, a GPI-anchor, and an N-acyl diglyceride group) are preferred embodiments of the invention, especially since the provision of such a lipidation anchor in a recombinantly produced protein is fairly straightforward and merely requires use of e.g. a naturally occurring signal sequence as a fusion partner for the modified EGFR polypeptide. Another possibility is use of the C3d fragment of complement factor C3 or C3 itself (cf. Dempsey et al., 1996, Science 271, 348-350 and Lou & Kohler, 1998, Nature Biotechnology 16, 458-462).

Another attractive way of presenting multiple copies of epitopic regions is the technology disclosed in WO 00/32227, where antigens are presented in ordered, repetitive patterns, thereby giving rise to T-cell independent immunogens that resemble virus capsids. In the context of the present invention, the technology of WO 00/32227 is regarded as application of a specialized adjuvant. The disclosure of WO 00/32227 is hereby incorporated by reference herein. An alternative embodiment of the invention which also results in the preferred presentation of multiple (e.g. at least 2) copies of the important epitopic regions of the EGFR polypeptide to the immune system is the covalent coupling of polyamino acids selected from the EGFR polypeptide, the subsequence thereof, or the analogues thereof to certain molecules and, when necessary, together with foreign T_{H} epitopes or one of the first, second or third moieties discussed above. For instance, polymers can be used, e.g. polyhydroxypolymers, notably carbohydrates such as dextran, cf. e.g. Lees A et al., 1994, Vaccine 12: 1160-1166; Lees A et al., 1990, J Immunol. 145: 3594-3600, but also mannose and mannan are useful alternatives. Integral membrane proteins from e.g. E. coli and other bacteria are also useful conjugation partners. The traditional carrier molecules such as keyhole limpet hemocyanin (KLH), tetanus toxoid, diphtheria toxoid, and bovine serum albumin (BSA) are also preferred and useful conjugation partners.

Considerations underlying chosen areas of introducing modifications in EGFR polypeptides are a) preservation of known and predicted B-cell epitopes, and b) preservation of 3D structure. At any rate, as discussed above, it is fairly easy to screen a set of modified EGFR molecules which have all been subjected to introduction of a T-cell epitope in different locations.

Since the invention involve immunization against of human EGFR, it is consequently preferred that the EGFR polypeptide discussed above is a human EGFR polypeptide—however, any discussions below of human EGFR could be used for EGFR from other species, notably those listed in the sequence listing of this application. It will then be understood that teachings relating to changes in the human sequence should be transposed to the relevant sequence in the relevant animal: From the sequence listing it appears where the boundaries for the mature EGFR peptide sequence can be found, ant it will be understood that any specific sequence data referred to in the human sequence should take offset in the corresponding sequences in the various mammalian EGFR sequences.

In the embodiments relating to human EGFR, it is especially preferred that the human EGFR polypeptide has been modified by substituting at least one amino acid sequence in SEQ ID NO: 1 with at least one amino acid sequence of equal or different length and containing a foreign T_{H} epitope. Alternatively, the foreign T_{H} epitope may simply be inserted in SEQ ID NO: 1.

More specifically, a T_{H} containing (or completing) amino acid sequence which is introduced into SEQ ID NO: 1 may be introduced at any amino acid in SEQ ID NO: 1. That is, the introduction is possible after any one of amino acids 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, and 621, in SEQ ID NO: 1, and, in case of an addition, also before amino acid 1. This may be accompanied by deletion of amino acid(s) 1 and/or 2 and/or 3 and/or 4 and/or 5 and/or 6 and/or 7 and/or 8 and/or 9 and/or 10 and/or 11 and/or 12 and/or 13 and/or 14 and/or 15 and/or 16 and/or 17 and/or 18 and/or 19 and/or 20 and/or 21 and/or 22 and/or 23 and/or 24 and/or 25 and/or 26 and/or 27 and/or 28 and/or 29 and/or 30 and/or 31 and/or 32 and/or 33 and/or 34 and/or 35 and/or 36 and/or 37 and/or 38 and/or 39 and/or 40 and/or 41 and/or 42 and/or 43 and/or 44 and/or 45 and/or 46 and/or 47 and/or 48 and/or 49 and/or 50 and/or 51 and/or 52 and/or 53 and/or 54 and/or 55 and/or 56 and/or 57 and/or 58 and/or 59 and/or 60 and/or 61 and/or 62 and/or 63 and/or 64 and/or 65 and/or 66 and/or 67 and/or 68 and/or 69 and/or 70 and/or 71 and/or 72 and/or 73 and/or 74 and/or 75 and/or 76 and/or 77 and/or 78 and/or 79 and/or 80 and/or 81 and/or 82 and/or 83 and/or 84 and/or 85 and/or 86 and/or 87 and/or 88 and/or 89 and/or 90 and/or 91 and/or 92 and/or 93 and/or 94 and/or 95 and/or 96 and/or 97 and/or 98 and/or 99 and/or 100 and/or 101 and/or 102 and/or 103 and/or 104 and/or 105 and/or 106 and/or 107 and/or 108 and/or 109 and/or 110 and/or 111 and/or 112 and/or 113 and/or 114 and/or 115 and/or 116 and/or 117 and/or 118 and/or 119 and/or 120 and/or 121 and/or 122 and/or 123 and/or 124 and/or 125 and/or 126 and/or 127 and/or 128 and/or 129 and/or 130 and/or 131 and/or 132 and/or 133 and/or 134 and/or 135 and/or 136 and/or 137 and/or 138 and/or 139 and/or 140 and/or 141 and/or 142 and/or 143 and/or 144 and/or 145 and/or 146 and/or 147 and/or 148 and/or 149 and/or 150 and/or 151 and/or 152 and/or 153 and/or 154 and/or 155 and/or 156 and/or 157 and/or 158 and/or 159 and/or 160 and/or 161 and/or 162 and/or 163 and/or 164 and/or 165 and/or 166 and/or 167 and/or 168 and/or 169 and/or 170 and/or 171 and/or 172 and/or 173 and/or 174 and/or 175 and/or 176 and/or 177 and/or 178 and/or 179 and/or 180 and/or 181 and/or 182 and/or 183 and/or 184 and/or 185 and/or 186 and/or 187 and/or 188 and/or 189 and/or 190 and/or 191 and/or 192 and/or 193 and/or 194 and/or 195 and/or 196 and/or 197 and/or 198 and/or 199 and/or 200 and/or 201 and/or 202 and/or 203 and/or 204 and/or 205 and/or 206 and/or 207 and/or 208 and/or 209 and/or 210 and/or 211 and/or 212 and/or 213 and/or 214 and/or 215 and/or 216 and/or 217 and/or 218 and/or 219 and/or 220 and/or 221 and/or 222 and/or 223 and/or 224 and/or 225 and/or 226 and/or 227 and/or 228 and/or 229 and/or 230 and/or 231 and/or 232 and/or 233 and/or 234 and/or 235 and/or 236 and/or 237 and/or 238 and/or 239 and/or 240 and/or 241 and/or 242 and/or 243 and/or 244 and/or 245 and/or 246 and/or 247 and/or 248 and/or 249 and/or 250 and/or 251 and/or 252 and/or 253 and/or 254 and/or 255 and/or 256 and/or 257 and/or 258 and/or 259 and/or 260 and/or 261 and/or 262 and/or 263 and/or 264 and/or 265 and/or 266 and/or 267 and/or 268 and/or 269 and/or 270 and/or 271 and/or 272 and/or 273 and/or 274 and/or 275 and/or 276 and/or 277 and/or 278 and/or 279 and/or 280 and/or 281 and/or 282 and/or 283 and/or 284 and/or 285 and/or 286 and/or 287 and/or 288 and/or 289 and/or 290 and/or 291 and/or 292 and/or 293 and/or 294 and/or 295 and/or 296 and/or 297 and/or 298 and/or 299 and/or 300 and/or 301 and/or 302 and/or 303 and/or 304 and/or 305 and/or 306 and/or 307 and/or 308 and/or 309 and/or 310 and/or 311 and/or 312 and/or 313 and/or 314 and/or 315 and/or 316 and/or 317 and/or 318 and/or 319 and/or 320 and/or 321 and/or 322 and/or 323 and/or 324 and/or 325 and/or 326 and/or 327 and/or 328 and/or 329 and/or 330 and/or 331 and/or 332 and/or 333 and/or 334 and/or 335 and/or 336 and/or 337 and/or 338 and/or 339 and/or 340 and/or 341 and/or 342 and/or 343 and/or 344 and/or 345 and/or 346 and/or 347 and/or 348 and/or 349 and/or 350 and/or 351 and/or 352 and/or 353 and/or 354 and/or 355 and/or 356 and/or 357 and/or 358 and/or 359 and/or 360 and/or 361 and/or 362 and/or 363 and/or 364 and/or 365 and/or 366 and/or 367 and/or 368 and/or 369 and/or 370 and/or 371 and/or 372 and/or 373 and/or 374 and/or 375 and/or 376 and/or 377 and/or 378 and/or 379 and/or 380 and/or 381 and/or 382 and/or 383 and/or 384 and/or 385 and/or 386 and/or 387 and/or 388 and/or 389 and/or 390 and/or 391 and/or 392 and/or 393 and/or 394 and/or 395 and/or 396 and/or 397 and/or 398 and/or 399 and/or 400 and/or 401 and/or 402 and/or 403 and/or 404 and/or 405 and/or 406 and/or 407 and/or 408 and/or 409 and/or 410 and/or 411 and/or 412 and/or 413 and/or 414 and/or 415 and/or 416 and/or 417 and/or 418 and/or 419 and/or 420 and/or 421 and/or 422 and/or 423 and/or 424 and/or 425 and/or 426 and/or 427 and/or 428 and/or 429 and/or 430 and/or 431 and/or 432 and/or 433 and/or 434 and/or 435 and/or 436 and/or 437 and/or 438 and/or 439 and/or 440 and/or 441 and/or 442 and/or 443 and/or 444 and/or 445 and/or 446 and/or 447 and/or 448 and/or 449 and/or 450 and/or 451 and/or 452 and/or 453 and/or 454 and/or 455 and/or 456 and/or 457 and/or 458 and/or 459 and/or 460 and/or 461 and/or 462 and/or 463 and/or 464 and/or 465 and/or 466 and/or 467 and/or 468 and/or 469 and/or 470 and/or 471 and/or 472 and/or 473 and/or 474 and/or 475 and/or 476 and/or 477 and/or 478 and/or 479 and/or 480 and/or 481 and/or 482 and/or 483 and/or 484 and/or 485 and/or 486 and/or 487 and/or 488 and/or 489 and/or 490 and/or 491 and/or 492 and/or 493 and/or 494 and/or 495 and/or 496 and/or 497 and/or 498 and/or 499 and/or 500 and/or 501 and/or 502 and/or 503 and/or 504 and/or 505 and/or 506 and/or 507 and/or 508 and/or 509 and/or 510 and/or 511 and/or 512 and/or 513 and/or 514 and/or 515 and/or 516 and/or 517 and/or 518 and/or 519 and/or 520 and/or 521 and/or 522 and/or 523 and/or 524 and/or 525 and/or 526 and/or 527 and/or 528 and/or 529 and/or 530 and/or 531 and/or 532 and/or 533 and/or 534 and/or 535 and/or 536 and/or 537 and/or 538 and/or 539 and/or 540 and/or 541 and/or 542 and/or 543 and/or 544 and/or 545 and/or 546 and/or 547 and/or 548 and/or 549 and/or 550 and/or 551 and/or 552 and/or 553 and/or 554 and/or 555 and/or 556 and/or 557 and/or 558 and/or 559 and/or 560 and/or 561 and/or 562 and/or 563 and/or 564 and/or 565 and/or 566 and/or 567 and/or 568 and/or 569 and/or 570 and/or 571 and/or 572 and/or 573 and/or 574 and/or 575 and/or 576 and/or 577 and/or 578 and/or 579 and/or 580 and/or 581 and/or 582 and/or 583 and/or 584 and/or 585 and/or 586 and/or 587 and/or 588 and/or 589 and/or 590 and/or 591 and/or 592 and/or 593 and/or 594 and/or 595 and/or 596 and/or 597 and/or 598 and/or 599 and/or 600 and/or 601 and/or 602 and/or 603 and/or 604 and/or 605 and/or 606 and/or 607 and/or 608 and/or 609 and/or 610 and/or 611 and/or 612 and/or 613 and/or 614 and/or 615 and/or 616 and/or 617 and/or 618 and/or 619 and/or 620 and/or 621 in SEQ ID NO: 1.

Certain exemplary embodiments utilises a modified EGFR polypeptide which is provided by introduction of a foreign T_{H} epitope in any one of the following regions of EGFR:
amino acids 80-96, where amino acids 502-621 of EGFR optionally are deleted (relevant when using the truncated template),
amino acids 101-108, where amino acids 502-621 of EGFR optionally are deleted (relevant when using the truncated template),
amino acids 162-163, where amino acids 502-621 of EGFR optionally are deleted (relevant when using the truncated template),
amino acids 204-220, where amino acids 502-621 of EGFR optionally are deleted (relevant when using the truncated template),
amino acids 244-260, where amino acids 502-621 of EGFR optionally are deleted (relevant when using the truncated template),
amino acids 288-301, where amino acids 502-621 of EGFR optionally are deleted (relevant when using the truncated template),
amino acids 311-313, where amino acids 502-621 of EGFR optionally are deleted (relevant when using the truncated template),
amino acids 318-338, where amino acids 502-621 of EGFR optionally are deleted (relevant when using the truncated template),
amino acids 458-474, where amino acids 502-621 of EGFR optionally are deleted (relevant when using the truncated template),
amino acid 501, where amino acids 502-621 of EGFR optionally are deleted (relevant when using the truncated template),
amino acids 572-588,
amino acids 572-591, and
amino acids 614-615,
   wherein the amino acid numbering conforms to that of SEQ ID NO: 1.

This embodiment may entail that the foreign T_{H} epitope is introduced
as an insertion preceding or following any one of the specified EGFR amino acids; or
as a substitution that includes deletion of any one or all the specified EGFR amino acids
as a C-terminal addition to amino acid 501 in the truncated template described herein.

Another embodiment of the present invention is the presentation of the EGFR analogues which do not include any subsequence of EGFR that binds productively to MHC class II molecules initiating a T-cell response.

The rationale behind such a strategy for design of the immunogen that engages the immune system to induce an anti-EGFR immune response is the following: It has been noted that when immunizing with autologous proteins formulated in an adjuvant which is sufficiently strong to break the body's tolerance towards the autologous protein, there is a danger that in some vaccinated individuals the immune response induced cannot be discontinued simply by discontinuing the immunisation. This is because the induced immune response in such individuals is most likely driven by a native T_{H} epitope of the autologous protein, and this has the adverse effect that the vaccinated individual's own protein will be able to function as an immunizing agent in its own right: An autoimmune condition has thus been established.

The preferred methods including use of foreign T_{H} epitopes have to the best of the inventors' knowledge never been observed to produce this effect, because the anti-self immune response is driven by a foreign T_{H} epitope, and it has been repeatedly demonstrated by the inventors that the induced immune response invoked by the preferred technology indeed declines after discontinuation of immunizations. However, in theory it could happen in a few individuals that the immune response would also be driven by an autologous T_{H} epitope of the relevant self-protein one immunises against)—this is especially relevant when considering self-proteins that are relatively abundant, whereas other therapeutically relevant self-proteins are only present locally or in so low amounts in the body, that a “self-immunization effect” is not a possibility; however, for EGFR, this effect cannot be excluded.

One very simple way of avoiding this self-immunisation is hence to altogether avoid inclusion in the immunogen of peptide sequences that could serve as T_{H} epitopes (and since peptides shorter than about 9 amino acids cannot serve as T_{H} epitopes, the use of shorter fragments is one simple and feasible approach). Therefore, this embodiment of the invention also serves to ensure that the immunogen does not include peptide sequences of the target EGFR that could serve as “self-stimulating T_{H} epitopes” including sequences that merely contain conservative substitutions in a sequence of the target protein that might otherwise function as a T_{H} epitope.

Preferred embodiments of the immune system presentation of the analogues of EGFR involve the use of a chimeric peptide comprising at least one EGFR derived peptide, which does not bind productively to MHC class II molecules, and at least one foreign T-helper epitope. Moreover, it is preferred that the EGFR derived peptide harbours a B-cell epitope. It is especially advantageous if the immunogenic analogue is one, wherein the amino acid sequences comprising one or more B-cell epitopes are represented either as a continuous sequence or as a sequence including inserts, wherein the inserts comprise foreign T-helper epitopes.

Again, such an embodiment is most preferred when the suitable B-cell epitope carrying regions of EGFR are constituted by short peptide stretches that in no way would be able to bind productively to an MHC Class II molecule. The selected B-cell epitope or -epitopes of EGFR should therefore comprise at most 9 consecutive amino acids of hEGFR at most 9 consecutive amino acids in SEQ ID NO: 1. Shorter peptides are preferred, such as those having at most 8, 7, 6, 5, 4, or 3 consecutive amino acids from the hEGFR amino acid sequence.

It is preferred that the analogue comprises at least one subsequence of SEQ ID NO: 1 so that each such at least one subsequence independently consists of amino acid stretches from EGFR selected from the group consisting of 9 consecutive amino acids, 8 consecutive amino acids, 7 consecutive amino acids, 6 consecutive amino acids, 5 consecutive amino acids, 4 consecutive amino acids, and 3 consecutive amino acids.

It is especially preferred that the consecutive amino acids begins at an amino acid residue selected from the group consisting of residue 1 and/or 2 and/or 3 and/or 4 and/or 5 and/or 6 and/or 7 and/or 8 and/or 9 and/or 10 and/or 11 and/or 12 and/or 13 and/or 14 and/or 15 and/or 16 and/or 17 and/or 18 and/or 19 and/or 20 and/or 21 and/or 22 and/or 23 and/or 24 and/or 25 and/or 26 and/or 27 and/or 28 and/or 29 and/or 30 and/or 31 and/or 32 and/or 33 and/or 34 and/or 35 and/or 36 and/or 37 and/or 38 and/or 39 and/or 40 and/or 41 and/or 42 and/or 43 and/or 44 and/or 45 and/or 46 and/or 47 and/or 48 and/or 49 and/or 50 and/or 51 and/or 52 and/or 53 and/or 54 and/or 55 and/or 56 and/or 57 and/or 58 and/or 59 and/or 60 and/or 61 and/or 62 and/or 63 and/or 64 and/or 65 and/or 66 and/or 67 and/or 68 and/or 69 and/or 70 and/or 71 and/or 72 and/or 73 and/or 74 and/or 75 and/or 76 and/or 77 and/or 78 and/or 79 and/or 80 and/or 81 and/or 82 and/or 83 and/or 84 and/or 85 and/or 86 and/or 87 and/or 88 and/or 89 and/or 90 and/or 91 and/or 92 and/or 93 and/or 94 and/or 95 and/or 96 and/or 97 and/or 98 and/or 99 and/or 100 and/or 101 and/or 102 and/or 103 and/or 104 and/or 105 and/or 106 and/or 107 and/or 108 and/or 109 and/or 110 and/or 111 and/or 112 and/or 113 and/or 114 and/or 115 and/or 116 and/or 117 and/or 118 and/or 119 and/or 120 and/or 121 and/or 122 and/or 123 and/or 124 and/or 125 and/or 126 and/or 127 and/or 128 and/or 129 and/or 130 and/or 131 and/or 132 and/or 133 and/or 134 and/or 135 and/or 136 and/or 137 and/or 138 and/or 139 and/or 140 and/or 141 and/or 142 and/or 143 and/or 144 and/or 145 and/or 146 and/or 147 and/or 148 and/or 149 and/or 150 and/or 151 and/or 152 and/or 153 and/or 154 and/or 155 and/or 156 and/or 157 and/or 158 and/or 159 and/or 160 and/or 161 and/or 162 and/or 163 and/or 164 and/or 165 and/or 166 and/or 167 and/or 168 and/or 169 and/or 170 and/or 171 and/or 172 and/or 173 and/or 174 and/or 175 and/or 176 and/or 177 and/or 178 and/or 179 and/or 180 and/or 181 and/or 182 and/or 183 and/or 184 and/or 185 and/or 186 and/or 187 and/or 188 and/or 189 and/or 190 and/or 191 and/or 192 and/or 193 and/or 194 and/or 195 and/or 196 and/or 197 and/or 198 and/or 199 and/or 200 and/or 201 and/or 202 and/or 203 and/or 204 and/or 205 and/or 206 and/or 207 and/or 208 and/or 209 and/or 210 and/or 211 and/or 212 and/or 213 and/or 214 and/or 215 and/or 216 and/or 217 and/or 218 and/or 219 and/or 220 and/or 221 and/or 222 and/or 223 and/or 224 and/or 225 and/or 226 and/or 227 and/or 228 and/or 229 and/or 230 and/or 231 and/or 232 and/or 233 and/or 234 and/or 235 and/or 236 and/or 237 and/or 238 and/or 239 and/or 240 and/or 241 and/or 242 and/or 243 and/or 244 and/or 245 and/or 246 and/or 247 and/or 248 and/or 249 and/or 250 and/or 251 and/or 252 and/or 253 and/or 254 and/or 255 and/or 256 and/or 257 and/or 258 and/or 259 and/or 260 and/or 261 and/or 262 and/or 263 and/or 264 and/or 265 and/or 266 and/or 267 and/or 268 and/or 269 and/or 270 and/or 271 and/or 272 and/or 273 and/or 274 and/or 275 and/or 276 and/or 277 and/or 278 and/or 279 and/or 280 and/or 281 and/or 282 and/or 283 and/or 284 and/or 285 and/or 286 and/or 287 and/or 288 and/or 289 and/or 290 and/or 291 and/or 292 and/or 293 and/or 294 and/or 295 and/or 296 and/or 297 and/or 298 and/or 299 and/or 300 and/or 301 and/or 302 and/or 303 and/or 304 and/or 305 and/or 306 and/or 307 and/or 308 and/or 309 and/or 310 and/or 311 and/or 312 and/or 313 and/or 314 and/or 315 and/or 316 and/or 317 and/or 318 and/or 319 and/or 320 and/or 321 and/or 322 and/or 323 and/or 324 and/or 325 and/or 326 and/or 327 and/or 328 and/or 329 and/or 330 and/or 331 and/or 332 and/or 333 and/or 334 and/or 335 and/or 336 and/or 337 and/or 338 and/or 339 and/or 340 and/or 341 and/or 342 and/or 343 and/or 344 and/or 345 and/or 346 and/or 347 and/or 348 and/or 349 and/or 350 and/or 351 and/or 352 and/or 353 and/or 354 and/or 355 and/or 356 and/or 357 and/or 358 and/or 359 and/or 360 and/or 361 and/or 362 and/or 363 and/or 364 and/or 365 and/or 366 and/or 367 and/or 368 and/or 369 and/or 370 and/or 371 and/or 372 and/or 373 and/or 374 and/or 375 and/or 376 and/or 377 and/or 378 and/or 379 and/or 380 and/or 381 and/or 382 and/or 383 and/or 384 and/or 385 and/or 386 and/or 387 and/or 388 and/or 389 and/or 390 and/or 391 and/or 392 and/or 393 and/or 394 and/or 395 and/or 396 and/or 397 and/or 398 and/or 399 and/or 400 and/or 401 and/or 402 and/or 403 and/or 404 and/or 405 and/or 406 and/or 407 and/or 408 and/or 409 and/or 410 and/or 411 and/or 412 and/or 413 and/or 414 and/or 415 and/or 416 and/or 417 and/or 418 and/or 419 and/or 420 and/or 421 and/or 422 and/or 423 and/or 424 and/or 425 and/or 426 and/or 427 and/or 428 and/or 429 and/or 430 and/or 431 and/or 432 and/or 433 and/or 434 and/or 435 and/or 436 and/or 437 and/or 438 and/or 439 and/or 440 and/or 441 and/or 442 and/or 443 and/or 444 and/or 445 and/or 446 and/or 447 and/or 448 and/or 449 and/or 450 and/or 451 and/or 452 and/or 453 and/or 454 and/or 455 and/or 456 and/or 457 and/or 458 and/or 459 and/or 460 and/or 461 and/or 462 and/or 463 and/or 464 and/or 465 and/or 466 and/or 467 and/or 468 and/or 469 and/or 470 and/or 471 and/or 472 and/or 473 and/or 474 and/or 475 and/or 476 and/or 477 and/or 478 and/or 479 and/or 480 and/or 481 and/or 482 and/or 483 and/or 484 and/or 485 and/or 486 and/or 487 and/or 488 and/or 489 and/or 490 and/or 491 and/or 492 and/or 493 and/or 494 and/or 495 and/or 496 and/or 497 and/or 498 and/or 499 and/or 500 and/or 501 and/or 502 and/or 503 and/or 504 and/or 505 and/or 506 and/or 507 and/or 508 and/or 509 and/or 510 and/or 511 and/or 512 and/or 513 and/or 514 and/or 515 and/or 516 and/or 517 and/or 518 and/or 519 and/or 520 and/or 521 and/or 522 and/or 523 and/or 524 and/or 525 and/or 526 and/or 527 and/or 528 and/or 529 and/or 530 and/or 531 and/or 532 and/or 533 and/or 534 and/or 535 and/or 536 and/or 537 and/or 538 and/or 539 and/or 540 and/or 541 and/or 542 and/or 543 and/or 544 and/or 545 and/or 546 and/or 547 and/or 548 and/or 549 and/or 550 and/or 551 and/or 552 and/or 553 and/or 554 and/or 555 and/or 556 and/or 557 and/or 558 and/or 559 and/or 560 and/or 561 and/or 562 and/or 563 and/or 564 and/or 565 and/or 566 and/or 567 and/or 568 and/or 569 and/or 570 and/or 571 and/or 572 and/or 573 and/or 574 and/or 575 and/or 576 and/or 577 and/or 578 and/or 579 and/or 580 and/or 581 and/or 582 and/or 583 and/or 584 and/or 585 and/or 586 and/or 587 and/or 588 and/or 589 and/or 590 and/or 591 and/or 592 and/or 593 and/or 594 and/or 595 and/or 596 and/or 597 and/or 598 and/or 599 and/or 600 and/or 601 and/or 602 and/or 603 and/or 604 and/or 605 and/or 606 and/or 607 and/or 608 and/or 609 and/or 610 and/or 611 and/or 612 and/or 613 and/or 614 and/or 615 and/or 616 and/or 617 and/or 618 and/or 619 and/or 620 and/or 619 in SEQ ID NO: 1, where this is possible given the length of the consecutive stretch.

### Formulation of EGFR and Modified EGFR Polypeptides

When effecting presentation of the EGFR polypeptide or the modified EGFR polypeptide to an animal's (in this case to a human's) immune system by means of administration of an immunogenically effective amount thereof, the formulation of the polypeptide follows the principles generally acknowledged in the art.

Preparation of Vaccines which Contain Peptide Sequences as Active Ingredients is Generally well understood in the art, as exemplified by U.S. Pat. Nos. 4,608,251; 4,601,903; 4,599,231; 4,599,230; 4,596,792; and 4,578,770, all incorporated herein by reference. Typically, such vaccines are prepared as injectables either as liquid solutions or suspensions; solid forms suitable for solution in, or suspension in, liquid prior to injection may also be prepared. The preparation may also be emulsified. The active immunogenic ingredient is often mixed with excipients which are pharmaceutically acceptable and compatible with the active ingredient. Suitable excipients are, for example, water, saline, dextrose, glycerol, ethanol, or the like, and combinations thereof. In addition, if desired, the vaccine may contain minor amounts of auxiliary substances such as wetting or emulsifying agents, pH buffering agents, or adjuvants which enhance the effectiveness of the vaccines; cf. the detailed discussion of adjuvants below.

The vaccines are conventionally administered parenterally, by injection, for example, either subcutaneously, intracutaneously, or intramuscularly. Additional formulations which are suitable for other modes of administration include suppositories and, in some cases, oral, buccal, sublinqual, intraperitoneal, intravaginal, anal, epidural, spinal, and intracranial formulations. For suppositories, traditional binders and carriers may include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10-95% of active ingredient, preferably 25-70%. For oral formulations, cholera toxin is an interesting formulation partner (and also a possible conjugation partner).

The polypeptides may be formulated into the vaccine as neutral or salt forms. Pharmaceutically acceptable salts include acid addition salts (formed with the free amino groups of the peptide) and which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 2,000 μg (even though higher amounts in the 1-10 mg range are contemplated), such as in the range from about 0.5 μg to 2,000 μg or 0.5 μg to 1,000 μg, preferably in the range from 1 μg to 500 μg and especially in the range from about 10 μg to 100 μg. Suitable regimens for initial administration and booster shots are also variable but are typified by an initial administration followed by subsequent inoculations or other administrations.

The manner of application may be varied widely. Any of the conventional methods for administration of a vaccine are applicable. These include oral application on a solid physiologically acceptable base or in a physiologically acceptable dispersion, parenterally, by injection or the like. The dosage of the vaccine will depend on the route of administration and will vary according to the age of the person to be vaccinated and the formulation of the antigen.

Some of the polypeptides of the vaccine are sufficiently immunogenic in a vaccine, but for some of the others the immune response will be enhanced if the vaccine further comprises an adjuvant substance.

Various methods of achieving adjuvant effect for the vaccine are known. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9, both of which are hereby incorporated by reference herein.

It is especially preferred to use an adjuvant which can be demonstrated to facilitate breaking of the autotolerance to autoantigens; in fact, this is essential in cases where unmodified EGFR is used as the active ingredient in the autovaccine. Non-limiting examples of suitable adjuvants are selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; γ-inulin; and an encapsulating adjuvant. In general it should be noted that the disclosures above which relate to compounds and agents useful as first, second and third moieties in the analogues also refer mutatis mutandis to their use in the adjuvant of a vaccine of the invention.

The application of adjuvants include use of agents such as aluminium hydroxide or phosphate (alum), commonly used as 0.05 to 0.1 percent solution in buffered saline, admixture with synthetic polymers of sugars (e.g. Carbopol®) used as 0.25 percent solution, aggregation of the protein in the vaccine by heat treatment with temperatures ranging between 70° to 101° C. for 30 second to 2 minute periods respectively and also aggregation by means of cross-linking agents are possible. Aggregation by reactivation with pepsin treated antibodies (Fab fragments) to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. Admixture with oils such as squalene and IFA is also preferred.

According to the invention DDA (dimethyldioctadecylammonium bromide) is an interesting candidate for an adjuvant as is DNA and γ-inulin, but also Freund's complete and incomplete adjuvants as well as quillaja saponins such as QuilA and QS21 are interesting as is RIBI. Further possibilities are monophosphoryl lipid A (MPL), the above mentioned C3 and C3d, and muramyl dipeptide (MDP).

Liposome formulations are also known to confer adjuvant effects, and therefore liposome adjuvants are preferred according to the invention.

Also immunostimulating complex matrix type (ISCOM® matrix) adjuvants are preferred choices according to the invention, especially since it has been shown that this type of adjuvants are capable of up-regulating MHC Class II expression by APCs. An ISCOM® matrix consists of (optionally fractionated) saponins (triterpenoids) from Quillaja saponaria, cholesterol, and phospholipid. When admixed with the immunogenic protein, the resulting particulate formulation is what is known as an ISCOM particle where the saponin constitutes 60-70% w/w, the cholesterol and phospholipid 10-15% w/w, and the protein 10-15% w/w. Details relating to composition and use of immunostimulating complexes can e.g. be found in the above-mentioned text-books dealing with adjuvants, but also Morein B et al, 1995, Clin. Immunother. 3: 461-475 as well as Barr I G and Mitchell G F, 1996, Immunol. and Cell Biol. 74: 8-25 (both incorporated by reference herein) provide useful instructions for the preparation of complete immunostimulating complexes.

Another highly interesting (and thus, preferred) possibility of achieving adjuvant effect is to employ the technique described in Gosselin et al., 1992 (which is hereby incorporated by reference herein). In brief, the presentation of a relevant antigen such as an antigen of the present invention can be enhanced by conjugating the antigen to antibodies (or antigen binding antibody fragments) against the Fcγ receptors on monocytes/macrophages. Especially conjugates between antigen and anti-FcγRI have been demonstrated to enhance immunogenicity for the purposes of vaccination.

Other possibilities involve the use of the targeting and immune modulating substances (i.a. cytokines) mentioned above as candidates for the first and second moieties in the modified versions of EGFR. In this connection, also synthetic inducers of cytokines like poly I:C are possibilities.

Suitable mycobacterial derivatives are selected from the group consisting of muramyl dipeptide, complete Freund's adjuvant, RIBI, and a diester of trehalose such as TDM and TDE.

Suitable immune targeting adjuvants are selected from the group consisting of CD40 ligand and CD40 antibodies or specifically binding fragments thereof (cf. the discussion above), mannose, a Fab fragment, and CTLA-4.

Suitable polymer adjuvants are selected from the group consisting of a carbohydrate such as dextran, PEG, starch, mannan, and mannose; a plastic polymer such as; and latex such as latex beads.

Yet another interesting way of modulating an immune response is to include the EGFR immunogen (optionally together with adjuvants and pharmaceutically acceptable carriers and vehicles) in a “virtual lymph node” (VLN) (a proprietary medical device developed by ImmunoTherapy, Inc., 360 Lexington Avenue, New York, N.Y. 10017-6501). The VLN (a thin tubular device) mimics the structure and function of a lymph node. Insertion of a VLN under the skin creates a site of sterile inflammation with an upsurge of cytokines and chemokines. T- and B-cells as well as APCs rapidly respond to the danger signals, home to the inflamed site and accumulate inside the porous matrix of the VLN. It has been shown that the necessary antigen dose required to mount an immune response to an antigen is reduced when using the VLN and that immune protection conferred by vaccination using a VLN surpassed conventional immunization using Ribi as an adjuvant. The technology is i.a. described briefly in Gelber C et al., 1998, “Elicitation of Robust Cellular and Humoral Immune Responses to Small Amounts of Immunogens Using a Novel Medical Device Designated the Virtual Lymph Node”, in: “From the Laboratory to the Clinic, Book of Abstracts, Oct. 12^{th}-15^{th} 1998, Seascape Resort, Aptos, Calif.”.

Microparticle formulation of vaccines has been shown in many cases to increase the immunogenicity of protein antigens and is therefore another preferred embodiment of the invention. Microparticles are made either as co-formulations of antigen with a polymer, a lipid, a carbohydrate or other molecules suitable for making the particles or the microparticles can be homogeneous particles consisting of only the antigen itself.

Examples of polymer based microparticles are PLGA and PVP based particles (Gupta R K et al., 1998) where the polymer and the antigen are condensed into a solid particle. Lipid based particles can be made as micelles of the lipid (so-called liposomes) entrapping the antigen within the micelle (Pietrobon P J, 1995). Carbohydrate based particles are typically made of a suitable degradable carbohydrate such as starch or chitosan. The carbohydrate and the anti-gen are mixed and condensed into particles in a process similar to the one used for polymer particles (Kas H S et al., 1997).

Particles consisting only of the antigen can be made by various spraying and freeze-drying techniques. Especially suited for the purposes of the present invention is the super critical fluid technology that is used to make very uniform particles of controlled size (York P, 1999 & Shekunov B et al., 1999).

It is expected that the vaccine should be administered at least once a year, such as at least 1, 2, 3, 4, 5, 6, and 12 times a year. More specifically, 1-12 times per year is expected, such as 1, 2, 3, 4, 5, 6, 7, 8, 9, 10, 11, or 12 times a year to an individual in need thereof. It has previously been shown that the memory immunity induced by the use of the preferred autovaccines according to the invention is not permanent, and therefore the immune system needs to be periodically challenged with the analogues.

Due to genetic variation, different individuals may react with immune responses of varying strength to the same polypeptide. Therefore, the vaccine according to the invention may comprise several different polypeptides in order to increase the immune response, cf. also the discussion above concerning the choice of foreign T-cell epitope introductions. The vaccine may comprise two or more polypeptides, where all of the polypeptides are as defined above.

The vaccine may consequently comprise 3-20 different modified or unmodified polypeptides, such as 3-10 different polypeptides. However, normally the number of polypeptides will be sought kept to a minimum such as 1 or 2 polypeptides.

As an alternative to the EGFR analogues of the invention, it is also possible to immunize by using anti-idiotypic antibodies or even mimotopes. The technologies for preparing anti-idiotypic antibodies that mimic an EGFR epitope are known in the art, but one especially interesting version involves use of autologous anti-idiotypic antibodies, which are reactive with an anti-EGFR antibody and which are modified by introduction of a foreign T helper epitope as generally described herein. Mimotopes can be isolated from libraries of random peptides that are screened in phage display against antibodies that bind EGFR specifically.

### Nucleic Acid Vaccination

As an alternative to classic administration of a peptide-based vaccine, the technology of nucleic acid vaccination (also known as “nucleic acid immunisation”, “genetic immunisation”, and “gene immunisation”) offers a number of attractive features.

First, in contrast to the traditional vaccine approach, nucleic acid vaccination does not require resource consuming large-scale production of the immunogenic agent (e.g. in the form of industrial scale fermentation of microorganisms producing modified EGFR). Furthermore, there is no need to device purification and refolding schemes for the immunogen. And finally, since nucleic acid vaccination relies on the biochemical apparatus of the vaccinated individual in order to produce the expression product of the nucleic acid introduced, the optimum posttranslational processing of the expression product is expected to occur; this is especially important in the case of autovaccination, since, as mentioned above, a significant fraction of the original EGFR B-cell epitopes should be preserved in the modified molecule, and since B-cell epitopes in principle can be constituted by parts of any (bio)molecule (e.g. carbohydrate, lipid, protein etc.). Therefore, native glycosylation and lipidation patterns of the immunogen may very well be of importance for the overall immunogenicity and this is expected to be ensured by having the host producing the immunogen.

Hence, a preferred embodiment of the invention comprises effecting presentation of modified EGFR to the immune system by introducing nucleic acid(s) encoding the modified EGFR into the animal's cells and thereby obtaining in vivo expression by the cells of the nucleic acid(s) introduced.

In this embodiment, the introduced nucleic acid is preferably DNA which can be in the form of naked DNA, DNA formulated with charged or uncharged lipids, DNA formulated in liposomes, DNA included in a viral vector, DNA formulated with a transfection-facilitating protein or polypeptide, DNA formulated with a targeting protein or polypeptide, DNA formulated with Calcium precipitating agents, DNA coupled to an inert carrier molecule, DNA encapsulated in a polymer, e.g. in PLGA (cf. the microencapsulation technology described in WO 98/31398) or in chitin or chitosan, and DNA formulated with an adjuvant. In this context it is noted that practically all considerations pertaining to the use of adjuvants in traditional vaccine formulation apply for the formulation of DNA vaccines. Hence, all disclosures herein which relate to use of adjuvants in the context of polypeptide based vaccines apply mutatis mutandis to their use in nucleic acid vaccination technology.

As for routes of administration and administration schemes of polypeptide based vaccines which have been detailed above, these are also applicable for the nucleic acid vaccines of the invention and all discussions above pertaining to routes of administration and administration schemes for polypeptides apply mutatis mutandis to nucleic acids. To this should be added that nucleic acid vaccines can suitably be administered intraveneously and intraarterially. Furthermore, it is well-known in the art that nucleic acid vaccines can be administered by use of a so-called gene gun, and hence also this and equivalent modes of administration are regarded as part of the present invention. Finally, also the use of a VLN in the administration of nucleic acids has been reported to yield good results, and therefore this particular mode of administration is particularly preferred.

Furthermore, the nucleic acid(s) used as an immunization agent can contain regions encoding the 1^{st}, 2^{nd} and/or 3^{rd} moieties, e.g. in the form of the immunomodulating substances described above such as the cytokines discussed as useful adjuvants. A preferred version of this embodiment encompasses having the coding region for the analogue and the coding region for the immunomodulator in different reading frames or at least under the control of different promoters. Thereby it is avoided that the analogue or epitope is produced as a fusion partner to the immunomodulator. Alternatively, two distinct nucleotide fragments can be used, but this is less preferred because of the advantage of ensured co-expression when having both coding regions included in the same molecule.

Accordingly, the invention also relates to a composition for inducing production of antibodies against EGFR, the composition comprising
a nucleic acid fragment or a vector of the invention (cf. the discussion of vectors below), and
a pharmaceutically and immunologically acceptable vehicle and/or carrier and/or adjuvant as discussed above.

Under normal circumstances, the EGFR variant-encoding nucleic acid is introduced in the form of a vector wherein expression is under control of a viral promoter. For more detailed discussions of vectors and DNA fragments according to the invention, cf. the discussion below. Also, detailed disclosures relating to the formulation and use of nucleic acid vaccines are available, cf. Donnelly J J et al, 1997, Annu. Rev. Immunol. 15: 617-648 and Donnelly J J et al., 1997, Life Sciences 60: 163-172. Both of these references are incorporated by reference herein.

### Live and Viral Vaccines

A third alternative for effecting presentation of modified EGFR to the immune system is the use of live vaccine technology. In live vaccination, presentation to the immune system is effected by administering, to the animal, a non-pathogenic microorganism which has been transformed with a nucleic acid fragment encoding a modified EGFR or with a vector incorporating such a nucleic acid fragment. The non-pathogenic microorganism can be any suitable attenuated bacterial strain (attenuated by means of passaging or by means of removal of pathogenic expression products by recombinant DNA technology), e.g. Mycobacterium bovis BCG., non-pathogenic Streptococcus spp., E. coli, Salmonella spp., Vibrio cholerae, Shigella, etc. Reviews dealing with preparation of state-of-the-art live vaccines can e.g. be found in Saliou P, 1995, Rev. Prat. 45: 1492-1496 and Walker PD, 1992, Vaccine 10: 977-990, both incorporated by reference herein. For details about the nucleic acid fragments and vectors used in such live vaccines, cf. the discussion below.

As an alternative to bacterial live vaccines, the nucleic acid fragment of the invention discussed below can be incorporated in a non-virulent viral vaccine vector such as a vaccinia strain (e.g. in a modified vaccinia Ankara, MVA) or any other suitable pox virus.

Normally, the non-pathogenic microorganism or virus is administered only once to the animal, but in certain cases it may be necessary to administer the microorganism more than once in a lifetime in order to maintain protective immunity. It is even contemplated that immunization schemes as those detailed above for polypeptide vaccination will be useful when using live or virus vaccines.

Alternatively, live or virus vaccination is combined with previous or subsequent polypeptide and/or nucleic acid vaccination. For instance, it is possible to effect primary immunization with a live or virus vaccine followed by subsequent booster immunizations using the polypeptide or nucleic acid approach.

The microorganism or virus can be transformed with nucleic acid(s) containing regions encoding the 1^{st}, 2^{nd} and/or 3^{rd} moieties, e.g. in the form of the immunomodulating substances described above such as the cytokines discussed as useful adjuvants. A preferred version of this embodiment encompasses having the coding region for the analogue and the coding region for the immunomodulator in different reading frames or at least under the control of different promoters. Thereby it is avoided that the analogue or epitopes are produced as fusion partners to the immunomodulator. Alternatively, two distinct nucleotide fragments can be used as transforming agents. Of course, having the 1^{st} and/or 2^{nd} and/or 3^{rd} moieties in the same reading frame can provide as an expression product, an analogue of the invention, and such an embodiment is especially preferred according to the present invention.

### Peptides, Polypeptides, and Compositions of the Invention

As will be apparent from the above, the present invention is based on the concept of immunising individuals against the EGFR antigen. The preferred way of obtaining such an immunization is to use modified versions of EGFR, thereby providing molecules which have not previously been disclosed in the art.

It is believed that the modified EGFR molecules discussed herein are inventive in their own right, and therefore an important part of the invention pertains to an EGFR analogue which is derived from an animal EGFR wherein is introduced a modification which has as a result that immunization of the animal with the analogue induces production of antibodies cross-reacting with the unmodified EGFR polypeptide. Preferably, the nature of the modification conforms with the types of modifications described above when discussing various embodiments of the method of the invention when using modified EGFR. Hence, any disclosure presented herein pertaining to modified EGFR molecules are relevant for the purpose of describing the EGFR analogues of the invention, and any such disclosures apply mutatis mutandis to the description of these analogues.

It should be noted that preferred modified EGFR molecules comprise modifications which results in a polypeptide having a sequence identity of at least 70% with EGFR or with a subsequence thereof of at least 10 amino acids in length. Higher sequence identities are preferred, e.g. at least 75% or even at least 80% or 85%. The sequence identity for proteins and nucleic acids can be calculated as (N_{ref}−N_{dif})·100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{dif}=2 and N_{ref}=8).

The invention also pertains to compositions useful in exercising the method of the invention. Hence, the invention also relates to an immunogenic composition comprising an immunogenically effective amount of an EGFR polypeptide which is a self-protein in an animal or a subsequence of such an EGFR polypeptide, said EGFR polypeptide or subsequence being formulated together with an immunologically acceptable adjuvant so as to break the animal's autotolerance towards the EGFR polypeptide, the composition further comprising a pharmaceutically and immunologically acceptable vehicle and/or carrier. In other words, this part of the invention pertains to the formulations of naturally occurring EGFR polypeptides/subsequences which have been described in connection with embodiments of the method of the invention.

The invention also relates to an immunogenic composition comprising an immunologically effective amount of an EGFR analogue defined above, said composition further comprising a pharmaceutically and immunologically acceptable diluent and/or vehicle and/or carrier and/or excipient and optionally an adjuvant. In other words, this part of the invention concerns formulations of modified EGFR, essentially as described hereinabove. The choice of adjuvants, carriers, and vehicles is accordingly in line with what has been discussed above when referring to formulation of modified and unmodified EGFR for use in the inventive method for the immunizing against autologous EGFR.

The polypeptides are prepared according to methods well-known in the art. Longer polypeptides are normally prepared by means of recombinant gene technology including introduction of a nucleic acid sequence encoding the EGFR analogue into a suitable vector, transformation of a suitable host cell with the vector, expression of the nucleic acid sequence, recovery of the expression product from the host cells or their culture supernatant, and subsequent purification and optional further modification, e.g. refolding or derivatization.

Shorter peptides are preferably prepared by means of the well-known techniques of solid- or liquid-phase peptide synthesis. However, recent advances in this technology has rendered possible the production of full-length polypeptides and proteins by these means, and therefore it is also within the scope of the present invention to prepare the long constructs by synthetic means.

### Nucleic Acid Fragments and Vectors of the Invention

It will be appreciated from the above disclosure that modified EGFR polypeptides can be pre-pared by means of recombinant gene technology but also by means of chemical synthesis or semisynthesis; the latter two options are especially relevant when the modification consists in coupling to protein carriers (such as KLH, diphtheria toxoid, tetanus toxoid, and BSA) and non-proteinaceous molecules such as carbohydrate polymers and of course also when the modification comprises addition of side chains or side groups to an EGFR polypeptide-derived peptide chain.

For the purpose of recombinant gene technology, and of course also for the purpose of nucleic acid immunization, nucleic acid fragments encoding modified EGFR are important chemical products. Hence, an important part of the invention pertains to a nucleic acid fragment which encodes an EGFR analogue, i.e. an EGFR derived polypeptide which either comprises the natural EGFR sequence to which has been added or inserted a fusion partner or, preferably an EGFR derived polypeptide wherein has been introduced a foreign T-cell epitope by means of insertion and/or addition, preferably by means of substitution and/or deletion. The nucleic acid fragments of the invention are either DNA or RNA fragments.

The nucleic acid fragments of the invention will normally be inserted in suitable vectors to form cloning or expression vectors carrying the nucleic acid fragments of the invention; such novel vectors are also part of the invention. Details concerning the construction of these vectors of the invention will be discussed in context of transformed cells and microorganisms below. The vectors can, depending on purpose and type of application, be in the form of plasmids, phages, cosmids, mini-chromosomes, or virus, but also naked DNA which is only expressed transiently in certain cells is an important vector. Preferred cloning and expression vectors of the invention are capable of autonomous replication, thereby enabling high copynumbers for the purposes of high-level expression or high-level replication for subsequent cloning.

The general outline of a vector of the invention comprises the following features in the 5′→3′ direction and in operable linkage: a promoter for driving expression of the nucleic acid fragment of the invention, optionally a nucleic acid sequence encoding a leader peptide enabling secretion (to the extracellular phase or, where applicable, into the periplasma) of or integration into the membrane of the polypeptide fragment, the nucleic acid fragment of the invention, and optionally a nucleic acid sequence encoding a terminator. When operating with expression vectors in producer strains or cell-lines it is for the purposes of genetic stability of the transformed cell preferred that the vector when introduced into a host cell is integrated in the host cell genome. In contrast, when working with vectors to be used for effecting in vivo expression in an animal (i.e. when using the vector in DNA vaccination) it is for security reasons preferred that the vector is not incapable of being integrated in the host cell genome; typically, naked DNA or non-integrating viral vectors are used, the choices of which are well-known to the person skilled in the art.

The vectors of the invention are used to transform host cells to produce the modified EGFR polypeptide of the invention. Such transformed cells, which are also part of the invention, can be cultured cells or cell lines used for propagation of the nucleic acid fragments and vectors of the invention, or used for recombinant production of the modified EGFR polypeptides of the invention. Alternatively, the transformed cells can be suitable live vaccine strains wherein the nucleic acid fragment (one single or multiple copies) have been inserted so as to effect secretion or integration into the bacterial membrane or cell-wall of the modified EGFR.

Preferred transformed cells of the invention are microorganisms such as bacteria (such as the species Escherichia [e.g. E. coli], Bacillus [e.g. Bacillus subtilis], Salmonella, or Mycobacterium [preferably non-pathogenic, e.g. M. bovis BCG]), yeasts (such as Saccharomyces cerevisiae), and protozoans. Alternatively, the transformed cells are derived from a multicellular organism such as a fungus, an insect cell, a plant cell, or a mammalian cell. Most preferred are cells derived from a human being, cf. the discussion of cell lines and vectors below. Recent results have shown great promise in the use of a commercially available Drosophila melanogaster cell line (the Schneider 2 (S₂) cell line and vector system available from Invitrogen) for the recombinant production of EGFR analogues of the invention, and therefore this expression system is particularly preferred. Also the spodoptera cells (SF cells) SF9 and SF21 are preferred.

For the purposes of cloning and/or optimised expression it is preferred that the transformed cell is capable of replicating the nucleic acid fragment of the invention. Cells expressing the nucleic fragment are preferred useful embodiments of the invention; they can be used for small-scale or large-scale preparation of the modified EGFR or, in the case of non-pathogenic bacteria, as vaccine constituents in a live vaccine.

When producing the modified EGFR of the invention by means of transformed cells, it is convenient, although far from essential, that the expression product is either exported out into the culture medium or carried on the surface of the transformed cell.

When an effective producer cell has been identified it is preferred, on the basis thereof, to establish a stable cell line which carries the vector of the invention and which expresses the nucleic acid fragment encoding the modified EGFR. Preferably, this stable cell line secretes or carries the EGFR analogue of the invention, thereby facilitating purification thereof.

In general, plasmid vectors containing replicon and control sequences which are derived from species compatible with the host cell are used in connection with the hosts. The vector ordinarily carries a replication site, as well as marking sequences which are capable of providing phenotypic selection in transformed cells. For example, E. coli is typically transformed using pBR322, a plasmid derived from an E. coli species (see, e.g., Bolivar et al., 1977). The pBR322 plasmid contains genes for ampicillin and tetracycline resistance and thus provides easy means for identifying transformed cells. The pBR plasmid, or other microbial plasmid or phage must also contain, or be modified to contain, promoters which can be used by the prokaryotic microorganism for expression.

Those promoters most commonly used in prokaryotic recombinant DNA construction include the B-lactamase (penicillinase) and lactose promoter systems (Chang et al., 1978; Itakura et al., 1977; Goeddel et al., 1979) and a tryptophan (trp) promoter system (Goeddel et al., 1979; EP-A-0 036 776). While these are the most commonly used, other microbial promoters have been discovered and utilized, and details concerning their nucleotide sequences have been published, enabling a skilled worker to ligate them functionally with plasmid vectors (Siebwenlist et al., 1980). Certain genes from prokaryotes may be expressed efficiently in E. coli from their own promoter sequences, precluding the need for addition of another promoter by artificial means.

In addition to prokaryotes, eukaryotic microbes, such as yeast cultures may also be used, and here the promoter should be capable of driving expression. Saccharomyces cerevisiae, or common baker's yeast is the most commonly used among eukaryotic microorganisms, although a number of other strains are commonly available. For expression in Saccharomyces, the plasmid YRp7, for example, is commonly used (Stinchcomb et al., 1979; Kingsman et al., 1979; Tschemper et al., 1980). This plasmid already contains the trpl gene which provides a selection marker for a mutant strain of yeast lacking the ability to grow in tryptophan for example ATCC No. 44076 or PEP4-1 (Jones, 1977). The presence of the trpl lesion as a characteristic of the yeast host cell genome then provides an effective environment for detecting transformation by growth in the absence of tryptophan.

Suitable promoting sequences in yeast vectors include the promoters for 3-phosphoglycerate kinase (Hitzman et al., 1980) or other glycolytic enzymes (Hess et al., 1968; Holland et al., 1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin of replication and termination sequences is suitable.

In addition to microorganisms, cultures of cells derived from multicellular organisms may also be used as hosts. In principle, any such cell culture is workable, whether from vertebrate or invertebrate culture. However, interest has been greatest in vertebrate cells, and propagation of vertebrate in culture (tissue culture) has become a routine procedure in recent years (Tissue Culture, 1973). Examples of such useful host cell lines are VERO and HeLa cells, Chinese hamster ovary (CHO) cell lines, and W138, BHK, COS-7 293, Spodoptera frugiperda (SF) cells (commercially available as complete expression systems from i.a. Protein Sciences, 1000 Research Parkway, Meriden, Conn. 06450, U.S.A. and from Invitrogen), and MDCK cell lines. In the present invention, an especially preferred cell line is S₂ available from Invitrogen, PO Box 2312, 9704 CH Groningen, The Netherlands.

Expression vectors for such cells ordinarily include (if necessary) an origin of replication, a promoter located in front of the gene to be expressed, along with any necessary ribosome binding sites, RNA splice sites, polyadenylation site, and transcriptional terminator sequences.

For use In mammalian cells, the control functions on the expression vectors are often provided by viral material. For example, commonly used promoters are derived from polyoma, Adenovirus 2, and most frequently Simian Virus 40 (SV40). The early and late promoters of SV40 virus are particularly useful because both are obtained easily from the virus as a fragment which also contains the SV40 viral origin of replication (Fiers et al., 1978). Smaller or larger SV40 fragments may also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

An origin of replication may be provided either by construction of the vector to include an exogenous origin, such as may be derived from SV40 or other viral (e.g., Polyoma, Adeno, VSV, BPV) or may be provided by the host cell chromosomal replication mechanism. If the vector is integrated into the host cell chromosome, the latter is often sufficient.

### Identification of Useful EGFR Analogues

It will be clear to the skilled person that not all variants or modifications of native EGFR will have the ability to elicit antibodies in an animal which are cross-reactive with the native form. It is, however, not difficult to set up an effective standard screen for modified EGFR molecules which fulfil the minimum requirements for immunological reactivity discussed herein. Hence, another part of the invention concerns a method for the identification of a modified EGFR polypeptide which is capable of inducing antibodies against unmodified EGFR in an animal species where the unmodified EGFR polypeptide is a self-protein, the method comprising preparing, by means of peptide synthesis or by molecular biological means, a set of mutually distinct modified EGFR polypeptides wherein amino acids have been added to, inserted in, deleted from, or substituted into the amino acid sequence of an EGFR polypeptide of the animal species thereby giving rise to amino acid sequences in the set which comprise T-cell epitopes which are and foreign to the animal species, or preparing a set of nucleic acid fragments encoding the set of mutually distinct modified EGFR polypeptides, testing members of the set for their ability to induce production of antibodies by the animal species against the unmodified EGFR, and identifying and optionally isolating the member(s) of the set which significantly induces antibody production against unmodified EGFR in the animal species, or identifying and optionally isolating the polypeptide expression products encoded by members of the set of nucleic acid fragments which significantly induces antibody production against unmodified EGFR polypeptide in the animal species.

In this context, the “set of mutually distinct modified EGFR polypeptides” is a collection of non-identical modified EGFR polypeptides which have e.g. been selected on the basis of the criteria discussed above (e.g. in combination with studies of circular dichroism, NMR spectra, and/or EGFR-ray diffraction patterns). The set may consist of only a few members but it is contemplated that the set may contain several hundred members. Likewise, the set of nucleic acid fragments is a collection of non-identical nucleic acid fragments, each encoding a modified EGFR polypeptide selected in the same manner.

The test of members of the set can ultimately be performed in vivo, but a number of in vitro tests can be applied which narrow down the number of modified molecules which will serve the purpose of the invention.

Since the goal of introducing the foreign T-cell epitopes is to support the B-cell response by T-cell help, a prerequisite is that T-cell proliferation is induced by the modified EGFR. T-cell proliferation can be tested by standardized proliferation assays in vitro. In short, a sample enriched for T-cells is obtained from a subject and subsequently kept in culture. The cultured T-cells are contacted with APCs of the subject which have previously taken up the modified molecule and processed it to present its T-cell epitopes. The proliferation of T-cells is monitored and compared to a suitable control (e.g. T-cells in culture contacted with APCs which have processed intact, native EGFR). Alternatively, proliferation can be measured by determining the concentration of relevant cytokines released by the T-cells in response to their recognition of foreign T-cells.

Having rendered highly probable that at least one modified EGFR of the set is capable of inducing antibody production against EGFR, it is possible to prepare an immunogenic composition comprising at least one modified EGFR polypeptide which is capable of inducing antibodies against unmodified EGFR in an animal species where the unmodified EGFR polypeptide is a self-protein, the method comprising admixing the member(s) of the set which significantly induces production of antibodies in the animal species which are reactive with EGFR with a pharmaceutically and immunologically acceptable carrier and/or vehicle and/or diluent and/or excipient, optionally in combination with at least one pharmaceutically and immunologically acceptable adjuvant.

Likewise, it is also possible to prepare an immunogenic composition which as an immunogen contains a nucleic acid fragment encoding an immunogenic EGFR analogue, cf. the discussion of nucleic acid vaccination above.

The above aspects of the invention are conveniently carried out by initially preparing a number of mutually distinct nucleic acid sequences or vectors of the invention, inserting these into appropriate expression vectors, transforming suitable host cells with the vectors, and expressing the nucleic acid sequences of the invention. These steps can be followed by isolation of the expression products. It is preferred that the nucleic acid sequences and/or vectors are prepared by methods comprising exercise of a molecular amplification technique such as PCR or by means of nucleic acid synthesis.

### Preamble to Examples

Briefly, the EGFR vaccination plan entails the following experimental tasks:

### Design and Construction of a Panel of Human EGFR Mutants, Including:

1) Acquiring a synthetic human EGFR encoding sequence (SEQ ID NO: 6)—can be purchased or synthesized.
2) Generating immunogenized hEGFR molecules by means of mutagenesis.
3) Expression of wild-type and immunogenized hEGFR molecules in insect cells or mammalian cells (such as S2, HI5, SF9, SF21, CHO, COS or HEK293).
4) Purification of the immunogenized hEGFR molecules.
Selection of hEGFR candidates, Including:
1) Immunization of mice/rabbits.

### 2) ELISA.

3) Experimenting with tumor growth in mice.
4) Assaying receptor binding.
5) Assaying tumor growth.
Testing of the hEGFR Variants In Vivo, Including:
1) Prophylactic and therapeutic experiments in nude mice xenografted with a human cancer cell line such as A431 or MDA.MB-468 by transfer of serum from immunized immunocompetent mice.
2) Prophylactic and therapeutic experiments in nude mice xenografted with a human cancer cell line such as A431 or MDA.MB-468 by transfer of serum from immunized hEGFR trans-genic mice.
Testing of the hEGFR In Vitro
1) Growth inhibition assay.
2) Receptor-binding assay.
Expression of Wild Type hEGFR in Insect Cells

Because of the relatively high molecular weight of EGFR protein and its relatively high degree of glycosylation and in order to facilitate purification by elimination of the refolding step, it has been decided to express the human EGFR molecules in insect cells, such as S2, HI5, SF9 or SF21 or in mammalian cells such as CHO or HEK293.

The expression vector p2ZOp2F (Hegedus et al., Gene. 1998 Jan. 30; 207(2):241-9) has been obtained. This vector carries a constitutive OpIE2 promoter. This vector encodes the secretion signal in order to export the recombinant proteins to the medium. The selection system of this vector is the ZEO resistance. All EGFR constructs were subcloned into this vector with an N-terminal or a C-terminal UNI HIS-tag used for purification.

Expression of Wild Type hEGFR in Mammalian Cells

An expression system using CHO (Chinese hamster ovary) cells will also be implemented for the final testing of selected molecules. wtEGFR will be produced for titer determinations of sera generated from the immunogenized molecules, since this molecule will have a structure and a glycosylation almost identical to the normal protein.

### Immunogenicity Assays

The variants will be tested with different adjuvants in animals to evaluate the potency of these for induction of an antibody response. These adjuvants could be either CFA/FIA, Adjuphos™, Alhydrogel™, QS21 or MF59, which have shown good efficacy in studies performed in the Assignee's laboratories on immunogenic variants of human Her2. Purified protein variants solubilised in an aqueous physiological compatible buffer will be formulated with the above-mentioned adjuvants.

Generation of Polyclonal Rat and Mice Sera Using wt-EGFR and EGFR-Variants

Each group of animals (rats and mice) will be immunized by sequential subcutaneously injections of a single purified variant formulated in one of the above-mentioned adjuvants. An amount of 100-500 μl in total will be transferred to each mouse or rat for each injection.

Establishment of an anti-hEGFR ELISA

EGFR reactive titers of the sera generated by immunization with the EGFR variants and wtEGFR will be determined in an ELISA. This ELISA will be set up by coating of polystyrene microtiter plates (Maxisorp, Nunc, Denmark) with purified wtEGFR expressed from insect cells or mammalian cells. Sera from immunized animals are then titrated in these coated plates followed by addition of an enzyme labeled secondary antiserum.

### Establishment of a Growth Inhibition Assay

A growth inhibitory assay has been established to measure the inhibitory ability of in vitro tumor cell growth by the generated antisera. This assay will be used to evaluate the potency of the individual constructs against each other. A431 cells or MDA-MB-468 cells are seeded in microtiterplates followed by incubation of sera in different dilutions made in serum from the same species. IgG fractions may be purified before use in this assay because of the growth stimulatory properties of serum. Growth is evaluated by subsequent (several days later) staining of adhered cells using methylene blue followed by measurement of OD620. This method is adapted from (ref, Modjtahedi)

### Establishment of a Receptor Binding Assay

A receptor binding assay will be established by using adhered whole cells mixed with sequential dilutions of generated antisera and a fixed amount of iodinated ligand, e.g., EGF or TGF-alpha. The binding assay is performed on ice, and residual binding on cells (after several washes) is determined by transfer of cells to Lumaplates (Packard) that are read in a Topcounter (Packard). This assay will be used to evaluate the potency of the individual constructs against each other.

### In Vivo EGFR Tumor Model

Nude mice have been the common model used for evaluating anti-EGFR monoclonal antibodies. A variety of human EGFR expressing cell lines have been used previously, among others the A431 and the MDA-MB-468 cell lines that we intend to use as the primary cell lines. These cell lines all overexpress EGFR

Different doses of A431 and MDA-MB-468 cells have been injected subcutaneously on the back in nude mice. A431 cells grow well in all tested doses: 1×106, 2.5×106, 5×106, 1×107 pr. mouse MDA-MB-468 needs to be injected in a dose of 5×106 pr mouse.

Since nude mice are immunocompromised, it is not possible to immunize these mice directly. Instead, immunocompetent mice or rats are immunized with the EGFR variants and the wt EGFR followed by transfer of sera from these mice to the nude mice. Both prophylactic and therapeutic experiments may be performed in nude mice depending on whether serum is transferred before or after inoculation of tumor cells. Cf. also Example 2.

Sera can be transferred from normal non-tolerant animals immunized with the variants. A variation of this animal model is to immunize hEGFR-transgenic mice instead of nontrans-genic mice and then transfer sera as described. Shering GmbH has developed such trans-genic mice.

### Example 1

### Design of EGFR Constructs

### Design of Constructs

EGFR is a 170 kDa transmembrane protein. The extracellular part is 110 kDa including carbohydrates and 70 kDa when it is deglycosylated. The extracellular part consists of four domains: L1 (domain I), CR1 (domain II), L2 (domain III), CR2 (domain IV). CR1 and CR2 are cysteine-rich domains that have a strong structural function. L1 and L2 are right handed β-helices, which is a helical structure formed by β-strands. The major ligands EGF and TGF-alpha bind to L2. This is also the domain to which several inhibiting mAbs bind.

Apart from the overexpression of normal EGFR, different deletional mutations have been observed in human malignancies. A truncated variant EGFRvIII is present in a high proportion of gliomas, breast, ovarian and, to a lesser extent, lung cancers. This variant lacks amino acid 6-273 and lacks therefore most of L1 and more than half of CR1. This variant cannot bind the ligands but it is constitutively active, and it is therefore important to design variants that will also inhibit this mutant-EGFR, e.g., by down-regulation.

### Template Design

Two wtEGFR templates were chosen: EGFR 1-621 and EGFR 1-501. EGFR 1-621 comprises the full-length extracellular domain of EGFR and EGFR 1-501 is a truncated version of this extracellular domain. The reasons for these choices are described below.

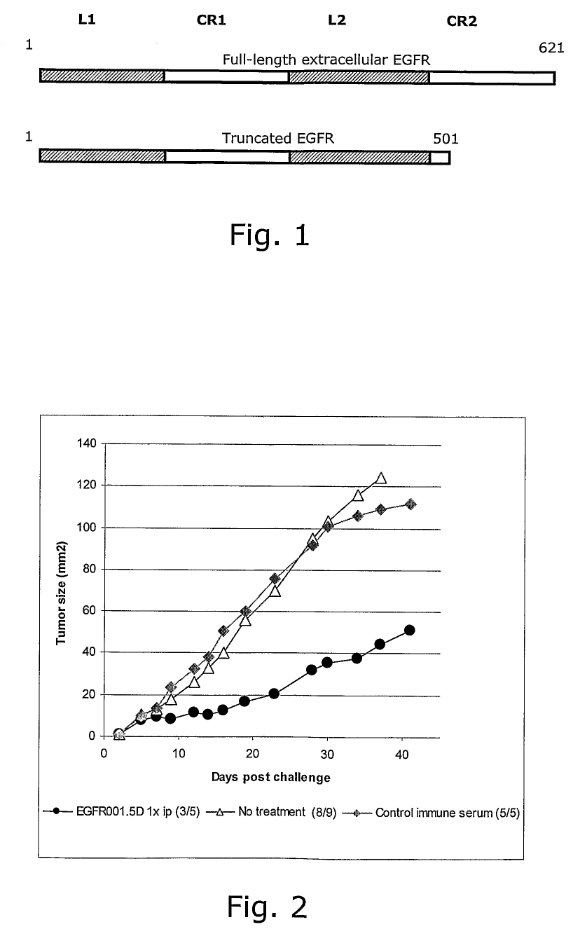

It is attempted to get a broad-spectred response toward different epitopes on EGFR. This is believed to be an advantage compared to monoclonal antibodies, since antibodies to different epitopes may have discrete ways of inhibiting EGFR-signaling. Furthermore, generated anti-sera should recognize both the normal EGFR and variant EGFRvIII in order to achieve an optimal effect. Therefore, the full-length extracellular part of EGFR is chosen as one of the templates (AA 1-621) (cf. FIG. 1).

Besides the full-length extracellular domain of EGFR, it has been decided also to use a truncated sequence, AA 1-501 (FIG. 1), since this sequence is known to bind the ligand with a higher affinity than the full-length extracellular part of EGFR. This is expected to lead to cross-reacting antibodies of a superior quality. Furthermore, it is expected to be easier to express molecules based on this template than molecules based on the full-length extracellular part.

The full-length extracellular domain of the wildtype EGFR is produced without a polyhistidinyl-tag and used for ELISA. It will be attempted to produce it in both insect cells and mammalian cells. It will also be produced with an N-terminal HIS-tag as a backup molecule. The truncated wtEGFR will not be produced in itself, only as variants with exemplary P2 (SEQ ID NO: 2) and P30 (SEQ ID NO: 3) insertions.

### Design of Variants

P2 and P30 (SEQ ID NOs: 2 and 3, respectively, and encoded by DNA fragments shown in SEQ ID NOs: 10 and 12, respectively), which are used as exemplary foreign T-helper epitopes, are substituted, partially substituted or inserted into the corresponding EGFR template (encoded by the relevant part of SEQ ID NO: 6). It is generally attempted to retain the natural structure as much as possible. Therefore, deduced flexible regions, regions with poor alignments within the family and experimentally identified sites have been used as modification sites.

All produced constructs are visualized in table 1. A description of the different insertion sites follows. Common for all molecules are an N-terminally placed UNI HIS-tag (SEQ ID NO: 5, encoded by SEQ ID NO: 8), except in EGFRs-5Dc where the HIS-tag is placed C-terminally (not stated in the table). Common to all constructs are also the substitution/insertion of both a P2 and a P30 sequence; it will be understood that this choice has been made for exemplification only and that use of one single very broad-spectred T-helper epitope (e.g. a PADRE such as SEQ ID NO: 4) will suffice to render a construct immunogenic in a broad population.

TABLE 1

| | last aa | first aa | | last aa | first aa | | |
|---|---|---|---|---|---|---|---|
| Construct | before p2 | after p2 | deleted by insert | before p30 | after p30 | deleted by insert | length |
| EGFR-4D | 204 | 220 | SDCCHNQCAAGCTGP | 614 | 615 | — | 642 |
| EGFR-5D | 244 | 260 | LYNPTTYQMDVNPEG | 614 | 615 | — | 642 |
| EGFR-6D | 318 | 334 | GEFKDSLSINATNIK | 614 | 615 | — | 642 |
| EGFR-9D | 572 | 588 | AGVMGENNTLVWKYA | 614 | 615 | — | 642 |
| EGFR-1 | 244 | 260 | LYNPTTYQMDVNPEG | 572 | 591 | AGVMGENNTLVWKYADAG | 624 |
| EGFR-2 | 101 | 108 | DANKTG | 244 | 260 | LYNPTTYQMDVNPEG | 637 |
| EGFR-3H | 311 | 313 | V | 572 | 591 | AGVMGENNTLVWKYADAG | 638 |
| EGFR-4 | 288 | 301 | ADSYEMEEDGVR | 614 | 615 | — | 645 |
| EGFR-5 | 458 | 474 | TSGQKIISNRGEN | 614 | 615 | — | 642 |
| EGFR-6 | 614 | 615 | — | 244 | 260 | LYNPTTYQMDVNPEG | 642 |
| EGFR-7 | 572 | 588 | AGVMGENNTLVWKYA | 244 | 260 | LYNPTTYQMDVNPEG | 627 |
| EGFR-8A | 80 | 96 | QIIRGNMYYENSYAL | 614 | 615 | — | 642 |
| EGFR-9L | 162 | 163 | — | 614 | 615 | — | 657 |
| EGFRs-3H | 311 | 313 | V | 501 | — | — | 522 |
| EGFRs-5Dn | 244 | 260 | LYNPTTYQMDVNPEG | 501 | — | — | 522 |
| EGFRs-5Dc | 244 | 260 | LYNPTTYQMDVNPEG | 501 | — | — | 522 |
| EGFRs-8A | 80 | 96 | QIIRGNMYYENSYAL | 501 | — | — | 522 |

The numbers used are calculated from the N-terminal LEEKK of EGFR where L is No. 1 and 621 is S from the C-terminal PKIPS.

One category of constructs are based on the full-length extracellular domain. This accounts for EGFR-4D, EGFR-5D, EGFR-6D, EGFR-9D, EGFR-1, EGFR-2, EGFR-3H, EGFR-4, EGFR-5, EGFR-6, EGFR-7, EGFR-8A and EGFR-9L.

Another category is based on the truncated template (EGFR1-501). This accounts for EGFRs-5D, EGFRs-8A and EGFR-4H.

Thus, a number of different sites have been used for insertion of either P2 or P30. Uses of these sites in the individual constructs are visualised in table 1 and are further described below.

### Amino Acids 80-96

This region is substituted by the P2 sequence. The sequence has been identified by the homology with the P2 sequence, which is 53% based on homology with conserved residues.

### Amino Acids 101-108

This region is partially substituted by the P2 sequence. The sequence has been identified since it represents a flexible region according to the published model on EGFR-structure.

### Amino Acids 162-163

This region is used for a complete insertion of the P2 sequence. A random insertion study of four amino acids has shown us that insertion in this region results in good expression and that the molecule retains binding to EGFR ligands.

### Amino Acids 204-220

This sequence is substituted by P2 and has been identified because of a similar successful substitution in the HER-2 molecule. Four cysteines are deleted.

### Amino Acids 244-260

This sequence is substituted by P2 or P30 and has been identified because of previously accomplished successful substitutions in the HER2-5D molecule. The sequence also represents a flexible region according to the published model of EGFR. Moreover, a random insertion study of four amino acids showed that insertion in this region resulted in good expression and good ligand binding.

### Amino Acids 288-301

This sequence is partially substituted with P2 and has been identified due to the existence of poor sequence homology and gaps in this region within members of the ErbB/IGF receptor family.

### Amino Acids 311-313

This sequence is used for complete insertion of the P2 sequence in the hinge region between the CR1 domain and the L2 domain of the EGFR.

### Amino Acids 318-338

This sequence is substituted with the P2 sequence and has been identified based on previous success with similar constructs based on the HER-2 molecule. This sequence is also identified as a flexible region according to the published structural model.

### Amino Acids 458-474

This region is partially substituted by the P2 sequence. The sequence has been identified due to the existence of poor sequence homology and gaps within this region in members of the ErbB/IGF receptor family

### Amino Acids 501

This site is the C-terminus of the truncated template. P30 is placed C-teminally after this residue in the truncated constructs.

### Amino Acids 572-588

This sequence is substituted by P2. The sequence has been identified as a consequence of the successful substitution in a corresponding region of HER-2. This site has also been identified as a good insertion site by the random insertion study. Furthermore, it partly represents a flexible region according to the published model.

### Amino Acids 572-591

This region is substituted by P30. The sequence has been identified because of a similar, successful substitution in the HER2 molecule. This site has also been identified as a good insertion site in a random insertion study. Furthermore, it partly represents a flexible region according to the published model.

### Amino Acids 614-615

This region is used for insertion of P30 in the C-terminus. The sequence is identified as a flexible region according to the published model. It is avoided to place P30 completely C-terminally because of the hydrophobic nature of the sequence.

### Example 2

### Effect of Passive Transfer of Anti-EGFR Antiserum Induced by Immunization with EGFR-5D

Immunocompetent mice were hyper-immunized with the EGFR-5D variant, and the antiserum was subsequently transferred to Nude mice challenged with A431 human tumor cells over expressing EGFR.

A single injection of 400 μl antiserum significantly delayed the growth of the tumors, as compared to non-treated mice and mice that received an injection of control antiserum. Cf. also FIG. 2.

1. A method for inducing an immune response against autologous Epidermal Growth Factor Receptor (EGFR) in a human subject, the method comprising effecting uptake and processing by antigen presenting cells (APCs) in the subject of at least one modified EGFR polypeptide, said at least one modified EGFR polypeptide comprising
a substantial fraction of the B-cell epitopes from the extracellular portion of human EGFR, and
at least one non-human T helper epitope (T_{H} epitope),
thereby inducing an antibody response that targets the autologous EGFR.

2. The method according to claim 1, for use in treatment or prophylaxis of neoplastic disease.

3. The method according to claim 1 or 2, wherein the modified human EGFR polypeptide comprises at least 60% of the 621 amino acids constituting the amino acid sequence of the extracellular domain of human EGFR.

4. The method according to claim 1, wherein the APC is a dendritic cell or a macrophage.

5. The method according to claim 1, wherein substantially all known epitopes of the extracellular portion of autologous EGFR are present in the first analogue and/or wherein substantially all predicted epitopes of the extracellular portion of autologous EGFR are present in the at least first analogue.

6. The method according to claim 1, wherein the modified human EGFR polypeptide can be provided by subjecting EGFR to amino acid substitution and/or deletion and/or insertion and/or addition.

7. The method according to claim 1, wherein the modified EGFR polypeptide comprises
at least one first moiety effecting targeting of the modified EGFR polypeptide to an antigen presenting cell (APC), and/or
at least one second moiety stimulating the immune system, and/or
at least one third moiety optimising presentation of the modified EGFR to the immune system.

8. The method according to claim 1, wherein the modified EGFR polypeptide includes duplication of at least one B-cell epitope of the autologous EGFR.

9. The method according to claim 1, wherein the at least one foreign T_{H} epitope is immunodominant and/or wherein the at least one foreign T_{H} epitope is promiscuous.

10. The method according to claim 1, wherein the modified EGFR polypeptide is provided by introduction of a foreign T_{H} epitope in any one of the following regions of EGFR:
amino acids 80-96, where amino acids 502-621 of EGFR optionally are deleted,
amino acids 101-108, where amino acids 502-621 of EGFR optionally are deleted,
amino acids 162-163, where amino acids 502-621 of EGFR optionally are deleted,
amino acids 204-220, where amino acids 502-621 of EGFR optionally are deleted,
amino acids 244-260, where amino acids 502-621 of EGFR optionally are deleted,
amino acids 288-301, where amino acids 502-621 of EGFR optionally are deleted,
amino acids 311-313, where amino acids 502-621 of EGFR optionally are deleted,
amino acids 318-338, where amino acids 502-621 of EGFR optionally are deleted,
amino acids 458-474, where amino acids 502-621 of EGFR optionally are deleted,
amino acid 501, where amino acids 502-621 of EGFR optionally are deleted,
amino acids 572-588,
amino acids 572-591, and
amino acids 614-615,
wherein the amino acid numbering conforms to that of SEQ ID NO: 1.

11. The method according to claim 10, wherein the foreign T_{H} epitope is introduced
as an insertion preceding or following any one of the specified EGFR amino acids; or
as a substitution that includes deletion of any one or all of the specified EGFR amino acids
as a C-terminal addition to amino acid 501 in an EGFR truncate where amino acids 502-621 are deleted.

12. The method according to claim 1, wherein foreign T_{H} epitope(s) is/are selected from a natural T_{H} epitope and an artificial MHC-II binding peptide sequence.

13. The method according to claim 12, wherein the natural T-cell epitope is selected from a Tetanus toxoid epitope, a diphtheria toxoid epitope, an influenza virus hemagluttinin epitope, and a P. falciparum CS epitope.

14. The method according to claim 1, wherein non-EGFR derived components such as foreign T_{H} epitopes of first, second and third moieties as defined in claim 7 are present in the form of
side groups attached covalently or non-covalently to suitable chemical groups in the amino acid sequence of the autologous EGFR or a subsequence thereof, and/or
fusion partners to the amino acid sequence derived from the autologous EGFR.

15. The method according to claim 14, wherein
the first moiety is a substantially specific binding partner for an APC specific surface antigen such as a carbohydrate for which there is a receptor on the APC, e.g. mannan or mannose, or wherein the first moiety is a hapten,
the second moiety is a cytokine selected from interferon y (IFN-y), Flt3L, interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF), or an effective part thereof; a heat-shock protein selected from heat shock protein 70 (HSP70), heat shock protein 90 (HSP90), heat shock cognate 70 (HSC70), glucose-regulated protein 94 (GRP94), and calrecticulin (CRT), or an effective part thereof; or a hormone,
the third moiety is a lipid such as a palmitoyl group, a myristyl group, a farnesyl group, a geranyl-geranyl group, a GPI-anchor, and an N-acyl diglyceride group.

16. The method according to claim 1, wherein the modified EGFR polypeptide substantially preserves the 3-dimensional structure of the EGFR extracellular domain.

17. The method according to claim 1, comprising administering an immunogenically effective amount of the at least one modified EGFR polypeptide.

18. The method according to claim 17, wherein said modified EGFR is formulated together with a pharmaceutically and immunologically acceptable carrier and/or vehicle and, optionally an adjuvant.

19. The method according to claim 18, wherein the adjuvant is selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; y-inulin; and an encapsulating adjuvant.

20. The method according to claim 19, wherein the cytokine is selected from interferon y (fFN-y), Flt3L, interleukin 1 (IL-1), interleukin 2 (IL-2), interleukin 4 (IL-4), interleukin 6 (IL-6), interleukin 12 (IL-12), interleukin 13 (IL-13), interleukin 15 (IL-15), and granulocyte-macrophage colony stimulating factor (GM-CSF), or an effective part thereof; a heat-shock protein selected from heat shock protein 70 (HSP70), heat shock protein 90 (HSP90), heat shock cognate 70 (HSC70), glucose-regulated protein 94 (GRP94), and calrecticulin (CRT), or an effective part thereof, or an effective part thereof, wherein the toxin is selected from the group consisting of listeriolycin (LLO), Lipid A (MPL, L180.5/RalLPS), and heat-labile enterotoxin, wherein the mycobacterial derivative is selected from the group consisting of muramyl dipeptide, complete Freund's adjuvant, RIBI, and a diester of trehalose such as TDM and TDE, wherein the immune targeting adjuvant is selected from the group consisting of CD40 ligand, CD40 antibodies or specifically binding fragments thereof, mannose, a Fab fragment, and CTLA-4, wherein the oil formulation comprises squalene or incomplete Fruend's adjuvant, wherein the polymer is selected from the group consisting of a carbohydrate such as dextran, PEG, starch, mannan, and mannose; a plastic polymer; and latex such as latex beads, wherein the saponin is Quillaja saponaria saponin, Quil A, and QS21, and wherein the particle comprises latex or dextran.

21. The method according to claim 17, which includes administration via a route selected from the oral route and the parenteral route such as the intracutaneous, the subcutaneous, the peritoneal, the buccal, the sublinqual, the epidural, the spinal, the anal, and the intracranial routes.

22. The method according to claim 17, which includes at least one administration a year, such as at least 2, 3, 4, 5, 6, and 12 administrations a year.

23. The method according to claim 1, comprising administering a non-pathogenic microorganism or virus which is carrying a nucleic acid fragment encoding and expressing the at least one modified EGFR polypeptide.

24. The method according to claim 23, wherein the non-pathogenic microorganism or virus is administered once to the animal.

25. The method according to claim 1, comprising administering, to the animal, at least one nucleic acid fragment which encodes and can express the at least one modified EGFR polypeptide.

26. The method according to claim 25, wherein the at least one nucleic acid fragment is selected from naked DNA, DNA formulated with charged or uncharged lipids, DNA formulated in liposomes, emulsified DNA, DNA inclided in a viral vector, DNA formulated with a transfection-facilitating protein or polypeptide, DNA formulated with a targeting protein or polypeptide, DNA formulated with a targeting carbohydrate, DNA formulated with Calcium precipitating agents, DNA coupled to an inert carrier molecule, and DNA formulated with an adjuvant.

27. The method according to claim 25, wherein the adjuvant is selected from the group consisting of the adjuvants defined in claim 19 or 20.

28. The method according to claim 25, wherein the mode of administration is as defined in claim 21 or 22.

29. A modified human EGFR polypeptide that is capable of inducing an immune response against autologous EGFR in a human subject, comprising a substantial fraction of the B-cell epitopes and optionally CTL epitopes from the extracellular portion of EGFR, and at least one non-human T helper epitope (T_{H} epitope).

30. The modified human EGFR polypeptide according to claim 29, which comprises
a substantial fraction of the B-cell epitopes from the extracellular portion of human EGFR, and
at least one non-human T helper epitope (T_{H} epitope).

31. An immunogenic composition which comprises, as an effective immunogenic agent the modified human EGFR according to claim 1 in admixture with a pharmaceutically and immunologically acceptable carrier or vehicle, and optionally an adjuvant.

32. A nucleic acid fragment which encodes a modified EGFR polypeptide according to claim 30.

33. A vector carrying the nucleic acid fragment according to claim 32.

34. The vector according to claim 33 being capable of autonomous replication.

35. The vector according to claim 33 or 34, being selected from the group consisting of a plasmid, a phage, a cosmid, a min-chromosome, and a virus.

36. The vector according to claim 33, comprising, in the 5′→3′ direction and in operable linkage, a promoter for driving expression of the nucleic acid fragment according to claim 32, optionally a nucleic acid sequence encoding a leader peptide enabling secretion of or integration into the membrane of the polypeptide fragment, the nucleic acid fragment according to claim 32, and optionally a nucleic acid sequence encoding a terminator.

37. The vector according to claim 33 which, when introduced into a host cell, is integrated in the host cell genome or is not capable of being integrated in the host cell genome.

38. A transformed cell carrying the vector of claim 33.

39. A composition for inducing production of antibodies against EGFR, the composition comprising
a nucleic acid fragment according to claim 32 or a vector according to claim 33, and
a pharmaceutically and immunologically acceptable diluent and/or vehicle and/or adjuvant.

40. A stable cell line which carries the vector according to claim 33 and which expresses the nucleic acid fragment according to claim 32, and which optionally secretes or carries the modified EGFR according to claim 30 on its surface.

41. A method for the preparation of the cell line according to claim 40, the method comprising transforming a host cell with the nucleic acid fragment according to claim 32 or with the vector according to claim 33.


SEQUENCE LISTING (13 sequences)

SEQ ID NO: 1 - protein, 621 aa, Homo sapiens
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 2 - protein, 15 aa, Clostridium tetani
QYIKANSKFIGITEL

SEQ ID NO: 3 - protein, 21 aa, Clostridium tetani
FNNFTVSFWLRVPKVSASHLE

SEQ ID NO: 4 - protein, 13 aa, Artificial sequence
  Pan DR binding epitope
AKFVAAWTLKAAA

SEQ ID NO: 5 - protein, 15 aa, Artificial sequence
  Non-natural His tag
MKHQHQHQHQHQHQQ

SEQ ID NO: 6 - DNA, 1869 bp, Homo sapiens
  CDS (1)..(1869)
atgctggaggaaaagaaagtttgccaaggcacgagtaacaagctcacgcagttgggcacttttgaagatcattttctcagcctccagaggatgttcaataactgtgaggtggtccttggaaatttggaaattacctatgtgcagaggaattatgatctttccttcctgaagaccatccaggaggtggctggttatgtcctcattgccctcaacacagtggagcgaattcctttggaaaacctgcagatcatccgcggaaatatgtactacgaaaattcctatgccttggcagtcctgtccaactatgatgcaaataaaaccggactgaaggagctgcccatgcgcaatttgcaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatggcagctgctggggtgcaggagaggagaactgccagaaactgaccaaaatcatctgtgcccagcagtgctccggacgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggagccgacagctatgagatggaggaagacggcgtccgcaagtgtaagaagtgcgaaggcccttgccgcaaagtgtgtaacggaataggtattggtgaatttaaagacagcctctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacaggctttttgctgattcaggcttggcctgaaaacaggacggacctccatgcctttgagaacctggaaatcatacgcggcaggaccaagcaacatggtcagttttcccttgcagtcgtcagcctgaacataacatccttgggactgcgctccctcaaggagataagtgatggagatgtgataatttcgggaaacaaaaatttgtgctatgcaaatacaataaactggaaaaaactgtttggcacctccggtcagaaaaccaaaattataagcaaccgcggtgaaaacagctgcaaggccacaggccaggtctgccatgccttgtgctcccccgagggctgctggggcccggagcccagggactgcgtctcgtgccggaatgtcagccgaggcagggaatgcgtggacaagtgcaaacttctggagggtgagccaagggagtttgtggagaactcggagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactggcccaggtcttgaaggctgtccaacgaatggacctaagatcccgtcctaa

SEQ ID NO: 7 - protein, 622 aa, Homo sapiens
MLEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 8 - DNA, 48 bp, Artificial sequence
  Synthetic DNA sequence encoding His tag
atgaagcaccaacaccaacatcaacatcaacatcaacatcaagccccc

SEQ ID NO: 9 - protein, 16 aa, Artificial sequence
  Synthetic Construct
MKHQHQHQHQHQHQAP

SEQ ID NO: 10 - DNA, 45 bp, Clostridium tetani
  CDS (1)..(45)
cagtacatcaaagctaactccaaattcatcggtatcactgaactg

SEQ ID NO: 11 - protein, 15 aa, Clostridium tetani
QYIKANSKFIGITEL

SEQ ID NO: 12 - DNA, 63 bp, Clostridium tetani
  CDS (1)..(63)
ttcaacaacttcaccgttagcttctggctgcgtgttccgaaagttagcgctagccacctggaa

SEQ ID NO: 13 - protein, 21 aa, Clostridium tetani
FNNFTVSFWLRVPKVSASHLE